SEQUENCE LISTING (8 sequences)

SEQ ID NO: 1 - DNA, 39 bp, Murine FGFR2c
attgaggttctctatattcggaatgtaacttttgaggat

SEQ ID NO: 2 - DNA, 40 bp, Modified murine FGFR2
attgaggttctctatattcggaatgtaagcttttgaggat

SEQ ID NO: 3 - DNA, 39 bp, Murine FGFR2
gatgctggggaatatacgtgcttggcgggtaattctatc

SEQ ID NO: 4 - DNA, 39 bp, Moeified murine FGFR2
gatgctggggaatatacgtacttggcgggtaattctatc

SEQ ID NO: 5 - protein, 13 aa, Murine FGFR2
IEVLYIRNVTFED

SEQ ID NO: 6 - protein, 11 aa, Modified murine FGFR2
IEVLYIRNVSF

SEQ ID NO: 7 - protein, 13 aa, Murine FGFR2
DAGEYTCLAGNSI

SEQ ID NO: 8 - protein, 13 aa, Modified murine FGFR2
DAGEYTYLAGNSI

(19) United States
(12) Patent Application Publication
(10) Pub. No.: US 2005/0239094 A1
(43) Pub. Date: Oct. 27, 2005

(54) Compositions and methods for inducing and regulating bone formation

(76) Inventors: Peter Lonai, Rehovot (IL); Rozmari Stirbu-Lonai, Rehovot (IL); Veraragavan P. Eswarakumar, New Haven, CT (US)

Correspondence Address: WINSTON & STRAWN LLP, 1700 K STREET, N.W., WASHINGTON, DC, 20006, US

(21) Appl. No.: 10/940,517

(22) Filed: Sep. 13, 2004

Related U.S. Application Data
(63) Continuation of application No. PCT/IL03/00203, filed on Mar. 12, 2003.
(60) Provisional application No. 60/430,073, filed on Dec. 2, 2002.

(30) Foreign Application Priority Data
Mar. 14, 2002 (IL) 148695

(51) Int. Cl. C12Q 1/68; C07H 21/04; C07K 14/71; C12N 15/09

(52) U.S. Cl. 435/6; 435/69.1; 435/320.1; 435/325; 530/350; 536/23.5

(57) ABSTRACT

The present invention relates to the regulation of bone formation, particularly to the regulation of bone formation by Fibroblast Growth Factor Receptor (FGFR) subtypes, and more particularly the FGFR2-IIIc subtype, to compositions containing active variants of FGFR2-IIIc, and to methods of using such compositions to treat bone and cartilage defects. Also, a non-human transgenic animal having an altered gene that includes a DNA encoding a gain of function mutation in FGFR2c, wherein the gain of function mutation results in the expression of an active variant of FGFR2c in the transgenic animal.

### CROSS-REFERENCE TO RELATED APPLICATIONS

This application is a continuation of International application PCT/IL03/00203 filed Mar. 12, 2003, and claims the benefit of U.S. provisional application 60/430,073 filed Dec. 2, 2002. The content of each application is expressly incorporated herein by reference thereto.

### FIELD OF THE INVENTION

The present invention relates to the regulation of bone formation, particularly to the regulation of bone formation by Fibroblast Growth Factor Receptor (FGFR) subtypes, more particularly the FGFR2-IIIC subtype, to compositions comprising active variants of FGFR2-IIIc, and to methods of using said compositions to treat bone and cartilage defects.

### BACKGROUND OF THE INVENTION

Numerous cell-regulating activities involve the signaling pathways triggered by activation of fibroblast growth factor receptors (FGFRs), specifically the FGFR subtypes FGFR1, FGFR2 and FGFR3, including pivotal roles in bone development. The three FGFR subtypes express splice variants, which alternatively use exons 8 or 9 to encode the C-terminal half of immunoglobulin-like loop III in their ligand binding domain. The IIIB-type receptors use exon 8 and bind FGFs that are mostly localized to the mesenchyme, whereas the IIIC-TYPE receptors preferentially use exon 9 and recognize epithelial FGFS.

Exon-specific gene targeting revealed that the activity of FGFR1 during late gastrulation is mostly due to its IIIC alternative. Recent studies imply that the IIIb alternative of FGFR1 may be active in skin development, serving as the receptor for the mesenchymal FGF10 growth factor.

Loss of FGFR2-IIIB abrogates limb outgrowth with multiple defects in branching morphogenesis. This phenotype is similar to the null mutation of FGFR2 after rescuing its placentation defects, and to the loss of function mutations of FGF10, the mesenchymal ligand of FGFR2-IIIB. The present invention is related to the IIIC subtype of FGFR2.

FGFR3 transcription localizes to the resting and proliferating chondrocyte layers and their loss results in long bone overgrowth with massive extension of the proliferating chondrocyte layer with enhanced type X collagen expression. Gain of function mutations of FGFR3, the cause of achondroplasia in man, creates the opposite phenotype. They repress collagen type X, Ihh and PTHRP signaling and activate cell cycle inhibitors thus indicating that FGFR3 decreases proliferation and increases differentiation in the chondrocyte lineage.

U.S. Pat. No. 6,517,872 discloses a culture comprising skeletal progenitor cells, that are obtained from a skeletal tissue and are enriched in vitro for cells that express FGFR3 on their surfaces and further discloses a pharmaceutical composition for the repair of bone and cartilage comprising the culture of these skeletal progenitor cells.

U.S. Pat. No. 6,447,783 discloses a method for stimulating cartilage or bone repair, comprising administering fibroblast growth factor 9 (FGF9) to a region of bone or cartilage requiring repair. The method may further comprise administering to the region heparin or a fragment of heparin with the ability to enhance binding of FGF9 to FGFR3.

U.S. Pat. No. 6,183,975 discloses a method of detecting a bone development disorder associated with a rhutation in a fibroblast growth factor receptor in a subject having an altered membrane component comprising: a) contacting a sample of cells obtained from the subject with a substance normally able to activate the membrane component in a wild type cell; and b) detecting an intracellular second messenger response after said contacting, wherein an abnormal second messenger response is indicative of the bone development disorder in the subject. Said bone development disorder is selected from the group consisting of achondroplasia, thanatophoric dysplasia type 1, thanatophoric dysplasia type 2, Crouzon, Jackson-Weiss, Pfeiffer and Apert syndrome, hypochondroplasia, Crouzon syndrome with acanthosis nigricans and fibroblast growth factor receptor 3-associated coronal synostosis.

U.S. patent application 20020009755 discloses a method based on the expression of IIIC isoform relative to the expression of the IIIb ISOFORM, of FGFR2, for monitoring the progression of prostate cancer.

International patent application WO 00/46343 discloses methods of screening for antagonist of FGFR-mediated malignant cell transformation using cells expressing the wild type and mutant variants of FGFR1, FGFR2 and FGFR3.

An FGFR2IIIc (−/−) loss of function (LOF) phenotype is disclosed by the present inventors and coworkers (Eswarakumar et al., 2002) published after the priority date of the present application, and is characterized by delayed onset of ossification, premature loss of skeletogenesis, with dwarfism in the long bones and axial skeleton. The retarded ossification in the FGFR2IIIc(−/−) phenotype was correlated with decrease in the localized transcription of the osteoblast markers secreted phosphoprotein 1 (Spp1) and Runx2/Cbfa1. A decrease in the domain of transcription of the chondrocyte markers, Ihh and PTHrP, was also demonstrated in this phenotype.

Nowhere in the background art is it taught or suggested that FGFR2-IIIc is the receptor directly related to osteogenesis, and that its biological activity is opposed to that of FGFR3.

### SUMMARY OF THE INVENTION

It is an object of the present invention to provide compositions for regulation of bone and cartilage formation and organization.

It is another object of the present invention to provide methods of regulating bone and cartilage formation, particularly, methods of treating diseases and disorders relating to defective bone growth, defective bone repair or healing, defective cartilage, including fracture healing in the old, osteoporosis, osteopetrosis, ostebarthritis, and inborn defects of osteogenesis.

The present invention is based in part on the unexpected finding of positive regulation of osteogenesis and fracture healing by FGFR2-IIIc, which is the mesenchymal splice variant of FGFR2, in gene targeting animal models.

According to the present invention it is now disclosed that FGFR2-IIIc is a positive regulator of osteogenesis. Moreover, its activity is contrary to that of FGFR3, a well-known FGFR subtype that acts as a negative regulator of osteogenesis.

The present invention discloses compositions and methods for treating diseases related to bone and cartilage defects, by activating FGFR2-IIIc. The present invention further discloses compositions and methods for treating diseases related to bone and cartilage defects, by activating FGFR2-IIIc and by inhibiting FGFR3. It is thus disclosed by the present invention that FGFR2-IIIc fulfills a positive role in bone development, which is in contrast to the known negative regulation by FGFR3.

Without wishing to be bound by any particular theory, the fact that these two opposed signaling pathways are critical for proper bone development suggests that individual steps of endochondral ossification are tightly coordinated. For example, signals from the joint region of the cartilage elements play an important role in regulating both chondrocyte proliferation and differentiation and at least some of these signals seem to interact with signals from the hypertrophic region, linking hypertrophic differentiation and proliferation. In addition, signals from the perichondriun/periosteum are thought to interact with signals from the differentiating chondrocytes to coordinate the differentiation of the periosteum with hypertrophic differentiation.

The compositions and methods of the present invention will be suitable for treating a broad spectrum of human diseases including but not limited to osteoporosis, osteopetrosis, osteoarthritis and achondroplasia. The bone defects that evolve in said diseases include fractures, altered bone density, bone and cartilage destruction and retarded development of the axial and appendicular skeleton.

According to one embodiment, the present invention provides an isolated polynucleotide encoding an active variant of FGFR2-IIIc.

According to a currently preferred embodiment, the present invention provides a constitutively active ligand-independent FGFR2-IIIc, based on exchange of Cysteine 342 to Tyrosine.

According to another embodiment, the present invention provides a construct comprising a polynucleotide sequence encoding an active variant of FGFR2-IIIc, preferably a constitutively active ligand-independent FGFR2-IIIc.

According to yet another embodiment, the present invention provides a vector comprising a polynucleotide sequence encoding an active variant of FGFR2-IIIc, preferably a constitutively active ligand-independent FGFR2-IIIc. The vector may further comprise a selectable promoter the activity of which is controlled by bone regulatory elements. The vector may be transfected into a host cell, therefore the invention further encompasses host cells expressing the molecules of the invention. For purposes of cell therapy, the vector may be stably integrated into host cell genomes. For these purposes, a currently preferred vector would be a viral vector, exemplified by but not limited to an adenovirus vector. The vector of the present invention may be also used for the purpose of gene therapy. Gene therapy may be accomplished by introducing a vector capable of expressing at least one of the following: FGFR2-IIIc and a ligand-independent FGFR2-IIIc, into a subject in need thereof.

According to yet another embodiment, the present invention provides a method of treating bone and cartilage defects and disorders by introducing the cells of the present invention, selected from a group of: somatic or hES cells, to a subject in need thereof. The cells of the invention may induce by cell therapy methods as are known in the art bone and cartilage formation. Cell therapy methods as are known in the art comprise transplanting into an individual in need thereof cells that have been genetically engineered and/or selected to express the required function. The cells may be genetically modified or selected in vitro for cell lineages that express the required function, said cells being capable of expressing at least one of the following entities: FGFR2-IIIc and a ligand-independent FGFR2-IIIc.

According to yet another embodiment, the present invention provides methods for increasing the expression of FGFR2-IIIc, or an active variant thereof, in cells of an individual in need thereof by use of gene therapy techniques as known in the art.

According to yet another embodiment, the present invention provides a pharmaceutical composition for regulating bone or cartilage growth or organization comprising as an active ingredient a vector comprising the polynucleotide sequence encoding an active variant of FGFR2c. The composition may further comprise at least one additional molecule selected from the group consisting of: an FGFR3 inhibitor, specific ligand for FGFR2-IIIc. Such molecules may be prepared by synthetic techniques, by recombinant techniques or may occur in nature. Said compositions may comprise a molecule that is an FGFR subtype-specific linase inhibitor.

According to yet another embodiment, the present invention provides a method of inducing bone and cartilage formation and organization by inducing FGFR2-IIIc activity by means of cell therapy or gene therapy, as described above, in conjunction with administration of a composition comprising at least one of the following entities: an FGFR3 inhibitor or an FGFR2-IIIc specific ligand.

The compositions disclosed in this invention may be in any pharmaceutical form suitable for administration to a patient, including but not limited to solutions, suspensions, lyophilized powders for reconstitution with a suitable vehicle, capsules and tablets. The pharmaceutical compositions disclosed in this invention may further comprise any pharmaceutically acceptable diluent or carrier.

The methods of the invention include the step of administering to a patient in need thereof an effective amount of the composition. Administration of the composition can be achieved by any appropriate route of administration, including but not limited to injecting the composition to the patient, inhalation, or implantation of a depot into the patient. The composition may further be administered by an osmotic pump. The osmotic pump can be implanted subcutaneously, or at any other appropriate site. Preferred sites may include sites close to the intended site of action, namely in proximity to the injured or diseased bone or cartilage.

These and other aspects of the present invention will be more fully understood from the drawings, detailed description and examples which follows.

### BRIEF DESCRIPTION OF THE DRAWINGS

FIG. 1 shows the genomic structure and targeting events required for the formation of a targeted disruption of the Fgfr2-IIIc transcriptional alternative (FIG. 1A). Exons are displayed as shaded boxes with their numbers given above and names beneath and wherein the following abbreviations are used: χ and γ: 5′ and 3′ probes respectively; B: BamHI; H: HindIII; RI: EcoRI; S: SacI; TM: trans membrane. # shows the site of point mutation. The DNA sequence of site directed mutagenesis (SEQ ID NO: 2) contains the newly formed HindIII site and a translational stop codon (FIG. 1B). Southern blot analysis of the homologous recombinant ES cells is shown in FIG. 1C.

FIG. 2 shows changes in the anatomy (FIG. 2A) using skeletal alizarin red staining (FIG. 2B) of homozygous FGFR2-IIIc mutants (IIIc−/−) and P14 wild type (+/+) mice and the corresponding growth retardation (FIG. 2C).

FIG. 3 shows bright and dark field views of in-situ hybridization for FGFR2-IIIb transcripts applied to wild type (+/+) and homozygous FGFR2-IIIc loss of function mutant (−/−) embryos at 14.5 days gestation in the limb bud (FIG. 3A) and in sagittal sections of the mid-trunk (FIG. 3B).

FIG. 4 shows the localization of FGFR2-IIIc transcripts in the limb bud at 12.5 days gestation (FIG. 4A) and in the skull at 18 days gestation (FIG. 4B). Localization of collagen type II (FIG. 4C) and collagen type X (FIG. 4D) is also shown in the skull base at 18 days gestation.

FIG. 5 demonstrates ossification in the skull base in the Fgfr2c^{-/-} loss of function (LOF) mutant and wild type at 16.5 days gestation (E16.5), 18.5 days gestation (E18.5).

FIG. 6 shows craniosynostosis in P14 FGFR2-IIIc mutants (−/−) and wild type by alizarin staining of the following skeletal preparations: the skull (FIGS. 6A-B); the coronal suture (FIG. 6C); Skull base—exoccipital, basioccipital and basisphenoid bones (FIG. 6D); supraoccipital and exoccipital bones (FIG. 6E).

FIG. 7 presents BrdU incorporation in wild type (FIG. 7A) and Fgfr2c^{-/-} LOF mutant (FIG. 7B) and Mallory-trichrome staining in the wild type (FIG. 7C) and Fgfr2c^{-/-} LOF mutant (FIG. 7D).

FIG. 8 shows a morphometric analysis of cell proliferation, evaluated by BrdU expression, in sections of the coronal suture (FIG. 8A), the hypertrophic zone (FIG. 8B; HZ) and the proliferating chondrocyte zones (FIG. 8B; PZ) from Fgfr2c^{-/-} mutants (MT) and wild type (WT).

FIG. 9 exhibits alizarin red stained skeletal bones at 14.5 days gestation (FIG. 9A) and first and second cervical vertebrae (FIG. 9B) at 14 days post-natal in LOF mutant and wild type. Hematoxylin/eosin-staining of tibia sections at 7 days post-natal is shown in LOF mutant and in the wild type (bc: bone collar; ep-oc: epiphysial ossification center; FIG. 9C).

FIG. 10 demonstrates osteopontin expression using whole mount in situ hybridization in the skull of E16.5 (FIG. 10A) and using radioactive in situ hybridization in the skull at 18.5 days gestation (FIGS. 10B-C) and in tibial growth plate of 15 days old neonates (FIG. 10D) mice.

FIG. 11 shows expression of chondrocyte and osteocyte markers in the Skull base at day 1 post-natal and tibia at day 7 post-natal (P7) of Fgfr2c^{-/-} LOF mutants (FIG. 11A) and wild type (FIG. 11B).

FIG. 12 shows the genomic structure and targeting events for targeted activation of the Fgfr2c transcriptional alternative (FIG. 12A; exons are shaded with the exon number above and the protein domain name underneath. X and Y, 3′ and internal probe, respectively). The DNA sequence of the region used for site-directed mutagenesis, showing the C342Y mutation and the newly formed RsaI site (SEQ ID NO: 4) is shown in FIG. 12B. FIG. 12C exhibits a southern blot analysis of the homologous recombination in ES cells, probed with the 3′ external probe after EcoRI digestion. Abbreviations: B, BamHI; H, HindIII; RI, EcoRI; S, Sacd; TM, transmembrane exon; #, site of point mutation.

FIG. 13 presents age matched wild type (FIG. 13A—top and FIG. 13B—right) with heterozygote (Fgfr2^{C342Y}; FIG. 13A bottom) and a homozygous mutant (FIG. 13B-left) and a wild type littermate (FIG. 13B-right) right after birth.

FIG. 14 demonstrates dorsal view (FIG. 14A) and ventral view (FIG. 14B) of alizarin stained skeletal preparations in 29 day old littermates Fgfr2^{C342Y/+} heterozygotes (right) as compared to wild type (left). Arrows point to the coronal and lambdoid sutures. Arrowheads indicate the basioccipital-basisphenoid and the basioccipital-exoccipital sutures. FIG. 14C represents Ssp1 expression (whole mount in situ hybridization) of the skull vault in E18.5 Fgfr2^{C342Y/+} heterozygote (right) as compared to wild type (left) fetuses.

FIG. 15 shows dorsal, ventral and lateral views of the naso-maxillary area and the overt cleft palate in the skull of homozygous Fgfr2^{C342Y/C342Y} gain-of-function (GOF) mutant (right) and wild type (left) newborn pups.

FIG. 16 demonstrates alizarin staining of knee joint (FIG. 16A), rib cage (FIG. 16B), vertebral bodies (FIG. 16C) tracheal rings (FIG. 16D) in Fgfr2^{C342Y/C342Y} gain-of-function (GOF) homozygous and in the wild type. FIG. 16E exhibits alizarin and alcian blue staining of the lung and trachea in the wild type, heterozygous and the homozygous Fgfr2^{C342Y/C342Y} mutant.

FIG. 17 shows Cbfa1 expression in mid-sagittal sections of the skull (FIG. 17A) and the basioccipital-basisphenoid junction (FIG. 17B) of E18.5 Fgfr2^{C342Y/C342Y} homozygotes mutant and wild type. FIG. 17C presents Cbfa1 expression in the humerus of E13.5 Fgfr2^{C342Y/C342Y} homozygotes mutant and in the wild type embryos.

FIG. 18 shows in situ hybridization for Ihh (FIG. 18A) and PTHrP (FIG. 18B) transcripts in the humerus of E13.5 wild type and Fgfr2^{C342Y/C342Y} homozygotes mutant embryos.

FIG. 19 demonstrates X-ray images of the tibiae of heterozygous mutant group (FIG. 19A) and control (wild type; FIG. 19B) group 3 weeks following a fracture procedure.

FIG. 20 exhibits H&E stained micro-sections from the tibiae of heterozygous mutant (FIG. 20B, FIG. 20D) and control (wild type; FIG. 20A, FIG. 20C) one week (FIGS. 20A-B) and five weeks (FIGS. 20C-D) following a fracture procedure.

FIG. 21 presents in situ hybridization with osteopontin (FIG. 21A) and osteocalcin (FIG. 21B) transcripts of bone micro-sections from Fgfr2c gain of function heterozygote mutants and wild type mice, at the site of experimental fraction.

### DETAILED DESCRIPTION OF THE INVENTION

The present invention discloses the positive regulation of osteogenesis by FGFR2-IIIc the mesenchymal splice variant of FGFR2. The activity of FGFR2-IIIc as a positive regulator of osteogenesis is contrary to that of FGFR3, the latter being a well-known FGFR subtype that acts as a negative regulator of osteogenesis.

Most bones of the mammalian skeleton are formed via endochondral ossification which is a multistep process regulated by a complex network of signaling systems. Endochondral ossification is initiated with the condensation of chondrocytes, cells that synthesize cartilage matrix, into cartilage elements in which the chondrocytes subsequently progress through stages of proliferation and hypertrophic differentiation. Finally, terminally differentiated chondrocytes undergo apoptosis and are replaced by bones formed from osteoblasts and their derivatives. Ihh and PTHrP are two signaling molecules that interact in a negative feedback loop regulating the pace of hypertrophic differentiation. In addition Ihh independently regulates chondrocytes proliferation and the ossification process, thus coordinating three different steps of endochondral bone formation.

The present invention in based in part on studies on the function of FGFR2-IIIc, wherein a translational stop codon was inserted into exon 9, which disrupted its synthesis without influencing the localized transcription of FGFR2IIIb, the epithelial FGFR2 variant. Recessive dwarfism with skull base synostosis characterized this mutation. Retarded ossification was observed in the entire skeleton, which was coupled with decrease in the localized transcription of osteoblast markers, such as osteopontin (Spp1) and Cbfa1. A certain decrease in the localized transcription of chondrocyte markers, such as Ihh and PTHrP, was also observed. FGFR2-IIIc is expressed in mesenchymal condensates and around the cartilage models, and later in the ossification zone of the growth plate. This expression pattern differs from that of FGFR3, which is transcribed in the zone of proliferating chondrocytes. We conclude that FGFR2-IIIc, in contrast to the negative role of FGFR3, is a positive regulator of ossification.

Gain of function mutation of FGFR3 causes achondroplasia in man and serious down-regulation of osteogenic factors in vitro. Thus, it is now disclosed that FGFR2c and FGFR3 fulfill opposite roles in osteogenesis, and that normal bone growth depends on a tightly regulated homeostasis between their activities.

Fracture healing in the old, osteoporosis, osteopetrosis, osteoarthritis, rheumatoid arthritis and inborn defects of osteogenesis are important clinical problems. Especially serious is involutional osteoporosis, which is the cause of many bone fractures in old people, especially in women, and it results in the USA in 1.5 million cases per annum. Fractures in the old frequently heal slowly or incompletely and their treatment is difficult, sometimes has debilitating results and can lead to embolism, which may be fatal. Osteoporosis has hereditary elements, but its etiology is poorly understood and no effective causal treatment is available. Osteoarthritis is another frequent and debilitating cartilage and bone disease, which commonly appears in middle and old age, leads to cartilage and finally bone loss and has no therapy. Any of the above mentioned bone and cartilage diseases and consequent defects would benefit from a therapy based on manipulating bone regulatory genes as disclosed in the present invention.

The present invention discloses methods and compositions for increasing the positive regulation of osteogenesis by FGFR2c and abrogating its negative control via FGFR3, in order to treat bone and cartilage diseases including osteoporosis, osteopetrosis, osteoarthritis and achondroplasia. It should be borne in mind that in children the bone growth plate is active until puberty and bone growth is thus achieved until puberty. Thus, treatment aimed at bone elongation, for example, by increasing the size of limb bones using methods within the scope of this invention, would be advantageous during this period.

The gain of function mutation of Fgfr2c enhances fracture healing. This mutation, i.e. the Cys342Tyr substitution, causing ligand-independent dimerization and activation of FGFR2 and craniosynostosis is associated with Crouzon syndrome in human (Mangasarian et al., 1997). Enhanced ossification and improved fracture healing was not reported in the context of Crouzon syndrome.

This was a powerful effect for two reasons. First, in heterozygotes the stable receptor dimers of the gain of function mutation are diluted with normal receptors, which decreases their effect beyond what could be expected from the homozygous mutation. Second, it is difficult to enhance normal, physiological processes to a significant extent. It is therefore expected that osteoblasts isolated from the homozygous mutant, or prepared in vitro from wild type osteoblasts or ES cells transfected with the gain of function construct, will have stronger effects. This experimental therapy will be extended to osteoporosis models, which we are establishing in mice.

The present invention also relates to methods of treatment of the various pathological conditions described above, by administering to a patient in need thereof a therapeutically effective amount of the compositions of the present invention. The term administration as used herein encompasses oral, parenteral, intravenous, intramuscular, subcutaneous, transdermal, and intralesional administration.

The present invention further relates to method for the use of the compositions of the invention to prepare medicaments useful of inducing fracture healing as well as in the treatment of various FGFR-related disorders including skeletal disorders such as achondroplasia and bone and cartilage defects such as osteoporosis, osteopetrosis, osteoarthritis.

We propose a therapy to support and enhance healing from bone-related diseases and symptoms including but not limited to fracture healing and induction of cartilage and bone growth using the role of Fgf2c as a positive regulator of ossification. Any of the applications suggested here, the use of the gain of function mutation (Cys342Tyr) of Fgfr2c via gene or cell therapy alone or in conjunction with inhibition of FGFR3 by a low molecular weight inhibitor should prove to be clinically applicable.

A. Definitions

For convenience certain terms employed in the specification, examples and claims are described herein:

The term “activate” as used herein refers to increasing or up regulating the function of the growth factor receptor.

The term “inhibit(s)” as used herein refers to decreasing or down regulating the function of the growth factor receptor.

The term “FGFR” as used herein refers to a fibroblast growth factor receptor encoded by a corresponding Fgfr gene and typically comprising an extracellular ligand-binding domain, a single transmembrane helix, and a cytoplasmic domain that contains a tyrosine kinase activity. The FGFR may be produced by cloning a polynucleotide sequence encoding the receptor in an expression vector. The present invention relates specifically to the receptor FGFR2-IIIc and the gene Fgfr2-IIIc encoding the receptor. The terms “FGFR2-IIIc” and “Fgfr2-IIIc” may be used interchangeably with the terms “FGFR2c” or “Fgfr2c”, respectively.

The term “gene targeting” as used herein refers to a process whereby a specific gene, or a fragment of that gene, is altered. This alteration of the targeted gene may result in a change in the level of RNA or protein that is encoded by that gene, or the alteration may result in the targeted gene encoding a different RNA or protein than the untargeted gene. The targeted gene may be studied in the context of a cell, or, more preferably, in the context of a transgenic animal. The gene that is targeted in the present invention is Fgfr2-IIIc and particularly segments of that gene denoted herein as SEQ ID NO: 1 and SEQ ID NO: 3 (Orr-Urtreger et al., 1993) resulting in mutagenic mice expressing a loss of function mutation or a gain of flunction mutation related to Fgfr2-IIIc also termed hereinafter Fgfr2c and the expressed receptor FGFR2-IIIc also denoted hereinafter FGFR2c.

A “loss of function” mutation refers to the inactivation of exon 9 in Fgfr2-IIIc. The term “Fgfr2c^{-/-}” or “FGFR2-IIIc(−/−)” are used interchangeably herein. Accordingly, the wild-type is also referred hereinafter “Fgfr2c^{+/+}”.

A “gain of function” or “GOF” mutation refers to a ligand independent receptor activation via the stabilization of receptor dimers of FGFR2-IIIc. This mutation is known as the Crouzon phenotype. Preferably, the mutation is achieved by converting the Cysteine 342 to Tyrosine. The gain of function (GOF) heterozygotes are also referred herein as “Fgfr2c^{C342Y/+} or “Fgfr2c^{gof/+}” accordingly the homozygotes are referred herein as “Fgfr2c^{C342Y/C342Y} or “Fgfr2c^{gof/gof}”.

The term “ligand-independent FGFR” as used herein refers to a stable fibroblast growth factor receptor dimer. A stable FGFR dimer may be attained by forming at least one inter-chain disulfide bridge. Receptor tyrosine kinases, such as FGFRs, function as dimers, therefore the dimerization results in a ligand-independent FGFR, which needs no ligand for signaling (phosphorylation) and is thus constitutively active.

The term “expression vector” as used herein refers to a recombinant DNA molecule containing a desired coding sequence and appropriate nucleic acid sequences necessary for the expression of the operably linked coding sequence in a particular host cell. It is contemplated that the present invention encompasses expression vectors that are integrated into host cell genomes, as well as vectors that remain unintegrated into the host genome.

The term “transfection” refers to the introduction of DNA into a host cell. It is contemplated that coding sequences may be expressed in transfected cells. Numerous methods of transfection are known to the ordinary skilled artisan, for example, CaPO₄ and electroporation.

A “specific ligand” as used herein is a molecule capable of being bound by the ligand-binding domain of a receptor particularly and selectively to FGFR2c. The ligands may be synthetic or prepared by recombinant techniques or may occur in nature. Binding of the ligands to a receptor induces a response pathway within a cell, including by a way of a non-limiting example, bone and cartilage formation and organization. Several ligands capable of specifically binding FGFR2c are known, for example: FGF2, FGF4, FGF6, FGF8, FGF9 and FGF1 (Ornitz et al., 1996).

The term “biological response of FGFR2c” as used herein refers to a cellular or physiologic response induced by the native receptor upon activation by ligands. Such response comprises upregulation of bone and cartilage formation, upregulation of positive regulators of the osteoblast and chondrocyte lineages, and induction of endochondral and intra membranous ossification.

The term “Polymerase Chain Reaction” (“PCR”) refers to the methods disclosed in U.S. Pat. Nos. 4,683,195; 4,683,202, and 4,965,188.

The term “genetically modified cells” as referred to herein relates to cells being transfected or infected by a vector, as exemplified by a virus encoding a polypeptide of interest, said cells capable of expressing said polypeptide. Particularly in the context of this invention, the genetically modified cells are capable of expressing at least one of the following: FGFR2-IIIc or ligand-independent FGFR2-IIIc. The cells subjected to genetic modifications are preferably human somatic cells, human embryonic stem (hES) cells or hES derived cells. The genetically modified somatic cells may in a certain embodiment be fibroblasts of the bone marrow. The genetically modified hES cells may be cultured under conditions that will direct them to differentiate into cells related to bone and cartilage, including but not limited to chondrocyte and osteoblast lineages.

B. Preferred Modes for Carrying Out the Invention

(i) FGFR2-IIIc Constitutive Dimer

The present invention encompasses an analog of FGFR2c monomer in which an amino acid substitution is incorporated producing a gain of function mutant. A most preferred embodiment of the present invention is an analog of FGFR2c in which Cysteine 342 located in the third Ig like domain of the receptor, is substituted with Tyrosine (SEQ ID NO: 8; Reardon W. et al., 1994). Said substitution disrupts an intra chain disulfide bond and creates a stable FGFR2c dimer which is capable of inducing the biological response of FGFR2c independently of ligand binding. A constitutively active FGFR2c stable dimer may also be formed by other methods known in the art. For example, using ligand-mimicking monoclonal antibodies directed to the native FGFR2c could generate an active dimer of the receptor, as has been observed in studies of other receptors that act as tyrosine kinases.

Methods for producing synthetic cDNA as described above through recombinant DNA technologies, are well known by those skilled in the art. Site directed mutagenesis is a preferred method for creating the ligand-independent Fgfr2c dimer construct using, for example, Polymerase Chain Reaction.

Natural or artificial substitutions of a single amino acid have been shown to form constitutively active FGFR mutants which cause skeletal disorders, e.g. Lys650Glu in FGFR3 results in strong constitutive activation of this receptor causing thanatophoric dysplasia type II. In Crouzon syndrome the Cys342Tyr substitution, resulting in ligand-independent dimerization and activation of FGFR2, causes craniosynostosis.

Nowhere in the background art is it taught or suggested that a stable dimer of FGFR2-IIIc, inducing constitutive activity, is used for osteogenesis, fracture healing and repair of bone and cartilage defects, as disclosed in the present invention. Importantly, upregulation of Fgfr2c independently of ligand binding increases the specifity of the biological response induced by this receptor, whereas ligands known to activate the natural FGFR2c monomer also activate other FGFRs, which may have an opposite response to that of Fgfr2c. For example, FGF10 activates both FGFR2c and FGFR3 whereas FGFR3 acts in an opposite manner to Fgfr2c on bone growth.

(ii) Cell Therapy

According to another embodiment, the present invention provides compositions for the purpose of treating bone and cartilage disorders by means of cell therapy, using cells expressing the molecules of the invention, namely FGFR2c or ligand-independent FGFR2c. According to yet another embodiment, the present invention provides methods for incorporation of oligonucleotides encoding the molecules of the invention into cells for cell therapeutic purposes.

Based on the available knowledge related to cell therapy, the cells preferred for usage as therapeutic tools are human embryonic stem cells (hES) or mesenchymal cells such as chondroblasts or fibroblasts which can be isolated from the subject or patient.

hES cells are defined as pluripotent cells derived from the inner cell, mass of blastocysts, with the capacity for unlimited proliferation in vitro in the undifferentiated state. These cells are capable of differentiating into many cell types and, therefore, they or their derivatives can be used for research and medical applications, including cellular transplantation. The characteristics expected of embryonic stem cells are: a) normal diploid karyotype, b) capacity for indefinite propagation in the undifferentiated state when grown on a feeder layer, c) telomerase enzyme activity in the undifferentiated state, d) formation of multicellular aggregates, yielding outgrowths containing multiple identifiable differentiated cell types, including derivatives of the three major germ cell layers (ectoderm, mesoderm, endoderm) upon release from the feeder layer. Using mesenchymal cells, such as fibroblasts, osteocytes, or chondrocytes, would provide alternatives to work with hES cells.

hES cells are allowed to differentiate under specific conditions chosen for selection and enrichment of the desired cell lineage. For example, hES cells of the present invention are selected to form cartilage, which is the basic tissue type forming the bone during the process of endochondral ossification. All long bones, the vertebral column, the rib cage and the skull base are formed by endochondral ossification. As is cormnonly known in the art, the conditions required for hES differentiation to a selective cell lineage include the use of varied cell growth factors, growth supplements, antioxidants or any other selected modifications to the culture medium that are known to predispose the cells to commit to a particular cell lineage. Various methods for generating novel embryonic cell populations, for propagation of embryonic stem cells utilizing combinations of growth factors and for immortalization of such cell lines are known in the art, as for example disclosed in the following patents: U.S. Pat. No. 5,690,926, EP 380646, and U.S. Pat. No. 6,110,739, among many others.

The preferred method for obtaining cells suitable for the purpose of cell therapy disclosed in the present invention, hES cells or mesenchymal cells are infected with vectors comprising the oligonucleotides encoding the molecules of the invention. Said vectors may contain selection genes that will enable selection of clones appropriately infected, as well known in the art. The preferred method of the present invention for selection of hES derived cell, relates to a positive selection scheme. Thus, a marker gene, such as a gene conferring antibiotic resistance (e.g. neomycin or hygromycin), is introduced into the stem cells under appropriate control such that expression of the gene occurs only in the desired cell lineage. For example, the marker gene can be under the control of a promoter which is active only in the desired cell linage. Upon differentiation of the stem cells, the desired lineage is then selected based upon the marker, e.g. by contacting the mixed cells with the appropriate antibiotic to which the desired lineage has been conferred resistance. Cell line other than the desired line will thus be killed, and substantially pure, homogeneous population of the desired line can be recovered. In more preferred methods, two markers are introduced into the parent stem cells, one allowing selection of vector-transfected stem cells from non-transfected cells, and one allowing selection of the desired cell lineage from other lineages. A double positive selection scheme can thus be used where each selectable marker confers antibiotic resistance. Using this selection methodology greatly enriches the populations of the desired cell linage.

According to yet another embodiment, the present invention provides methods for implanting said hES cells or mesenchymal cell clones into individuals in need thereof. The cells can be introduced in any suitable manner, but it is preferred that the mode of introduction be as non-invasive as possible. Thus, delivery of the cells by injection, catheterization or similar means will be more desired. It is contemplated that the transfected cells of the invention, for example hES cells directed to differentiate into chondrocytes, will join the differentiating blastema of the regenerating bone and cartilage upon damage thereof.

(iii) Gene Therapy

Another preferred embodiment of the present invention includes introduction of vectors, comprising polynucleotides that encode Fgfr2c or ligand-independent Fgfr2c, into a subject in need thereof. Cloning of said polynucleotides into expression vectors is known in the art. A preferred embodiment of the expression vectors is viral vectors, for example: adenoviruses, retroviruses or lentiviruses. The use of adenovirus vectors has been described, e.g. by (Cao et al., 1998). The use of SV40 derived viral vectors and SV-40 based packaging systems has been described by Fang et al. (Fang et al., 1997).

When using viruses as vectors, the viral surface proteins are generally used to target the virus. As many viruses, such as the above adenovirus, are somewhat unspecific in their cellular tropism, it may be desirable to impart further specificity by using a cell-type or tissue-specific promoter. Griscelli et al. (1998) teach the use of the ventricle-specific cardiac myosin light chain 2 promoter for heart-specific targeting of a gene whose transfer is mediated by adenovirus. For the purpose of the present invention the promoter activity may be controlled by factors specifically abundant in the bone and cartilage tissues.

Alternatively, the viral vector may be engineered to express an additional protein on its surface, or the surface protein of the viral vector may be changed to incorporate a desired peptide sequence. The viral vector may thus be engineered to express one or more additional epitopes which may be used to target said viral vector. For instance, cytokine epitopes, MHC class II-binding peptides, or epitopes derived from homing molecules may be used to target the viral vector in accordance with the teaching of the invention. Langner et al. (1998) teaches the use of heterologous binding motifs to target B-lymphotrophic papoaviruses.

A gene delivery composition comprising an FGFR is disclosed U.S. Pat. No. 6,503,886. The composition is having the formula: a polypeptide that binds to an FGFR-nucleic acid molecule, wherein the nucleic acid molecule being chemically conjugated or fused to the polypeptide and wherein the gene delivery composition binds to an FGFR and is internalized specifically in cells bearing the receptor.

Another method for a gene delivery to cultured cells is disclosed in U.S. Pat. No. 6,448,083. The method comprising: contacting a mammalian cell with filamentous phage particles presenting a ligand on their surfaces, wherein a vector within the phage encodes a gene product under control of a promoter. The ligand may be a polypeptide reactive with an FGF receptor, for example, FGF-2.

(iv) Other Approaches for Regulating Fgfr2c

Another embodiment of the present invention relates to activation of FGFR2-IIIc by specific ligands in conjunction with upregulation of Fgfr2c by gene and cell therapies as discussed above. There are inherent reasons why the approach relating to activation of FGFR2c by specific ligands by itself, may not be completely successful. Transcriptional alternatives of FGFR2 display specific localization. The IIIc variant is mostly expressed in mesenchymal tissues, whereas the IIIb variant mostly in epithelia. This is valid for positive regulators, of bone growth, such as Fgfr2c as well as for negative regulators, such as Fgfr3. It is therefore possible that systemic or local treatment with a specific ligand will activate receptors with positive effects as well as receptors with negative effects. Such dual activation could eliminate any therapeutic effect related to growth induction of bone and cartilage, the objects of this invention.

An additional embodiment of the present invention relates to inhibition of FGFR3 in conjunction with upregulation of Fgfr2c by gene and cell therapies as discussed above. FGFRs mediate their effects with intrinsic protein tyrosine kinase activity. A major issue of modern pharmacology is to devise small molecular weight inhibitors of kinase domains. Such is the Abl-kinase inhibitor Glivac, which is used successfully to treat myeloid leukemia. The structure of FGFR1 kinase domain is the basis for the design of numerous small molecular weight inhibitors against FGFRs. Although kinase domains are highly conserved, some of the inhibitors display a degree of subtype specificity. The addition of FGFR3 specific inhibitor to the therapies disclosed in this invention supports the induction of bone and cartilage growth by inhibiting the negative regulation of FGFR3 that opposes the positive regulation induced by FGFR2c.

A preferred embodiment of the present invention relates to the administration of FGFR2c specific ligands or FGFR3 inhibitors in the form of pharmaceutical compositions as will be elaborated hereinbelow.

(v) Pharmacology

The foremost therapeutic approaches of the present invention relate to cell therapy or gene therapy, wherein the pharmacological parameters of the compositions may have to be adapted individually to the subject in need of treatment. In other words, cell therapy may be accomplished with hES cells or hES derived cells, however, they may also utilize somatic cells, in which case the optimal choice would be the individual's autologous cells.

In addition to the novel polynucleotides, vectors and cells of the invention, treatment may include certain active ingredients that are polypeptides or srlall molecules namely, FGFR2c specific ligand or FGFR3 inhibitors. This consideration dictates that the formulation be suitable for delivery of these type of compounds. Clearly, polypeptides are less suitable for oral administration due to susceptibility to digestion by gastric acids or intestinal enzymes. It is contemplated that the present invention encompasses polypeptide compositions designed to circumvent these problems. The preferred routes of administration of polypeptides are intra-articular, intravenous, intramuscular, subcutaneous, intradermal, or intralesional. A more preferred route is by direct injection at or near the site of disorder or disease.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Pharmaceutical compositions, which can be used orally include push-fit capsules. All formulations for oral administration should be in dosages suitable for the chosen route of administration. For administration by inhalation, the molecules for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the polypeptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., 2001). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols. Pharmaceutical compositions may also include one or more additional active ingredients.

For bone and cartilage repair, administration may be preferred locally by means of a direct injection at or near the site of target or by means of a patch or subcutaneous implant, staples or slow release formulation implanted at or near the target.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate symptoms of a disease of the subject being treated. Determination of a therapeutically effective amount is well within the capability of those skilled in the art, especially in light of the detailed disclosure provided herein.

Toxicity and therapeutic efficacy of the peptides described herein can be determined by standard pharmaceutical procedures in cell cultures or experimental animals, e.g., by determining the IC₅₀ (the concentration which provides 50% inhibition) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

Having now generally described the invention, the same will be more readily understood through reference to the following examples, which are provided by way of illustration and are not intended to be limiting of the present invention.

### EXAMPLES

General Materials and Methods

In the following, general materials and methods used in the practice of the invention are described. A number of methods are not detailed herein as they are well known to the skilled artisan. These include genetic engineering techniques, site directed mutagenesis and the like. Techniques relating to molecular biology are detailed in many articles and textbooks, for instance, in (Sambrook et al., 1989). Likewise, techniques relating to histology, e.g. immunohistochemistry, are detailed in Kaufman (Kaufman, 1992).

Formation of Mutant FGF2-IIIc Constructs

A previously described genomic fragment including exons 7, 8, 9 and 10 (Arman et al, 1998) was used. For site directed mutagenesis the “Gene Editor” kit (Promega) was used according to the manufacturers instructions.

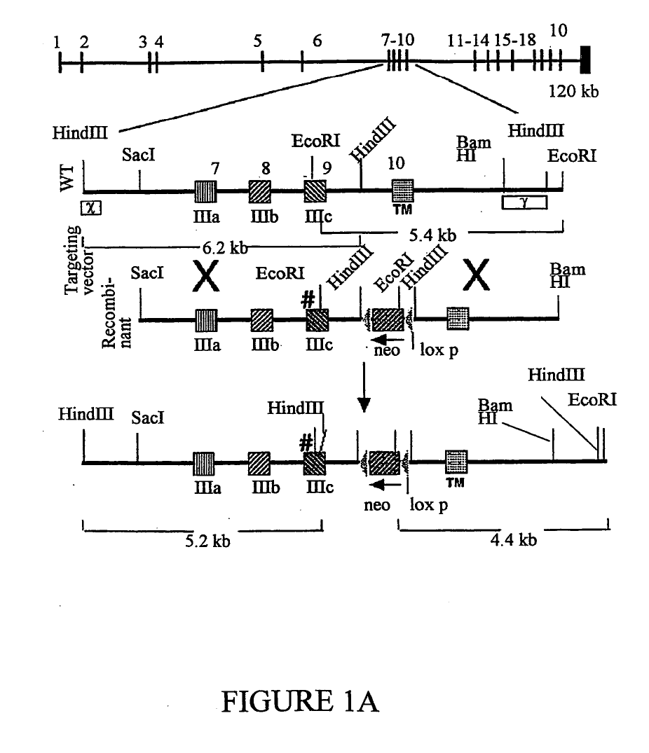

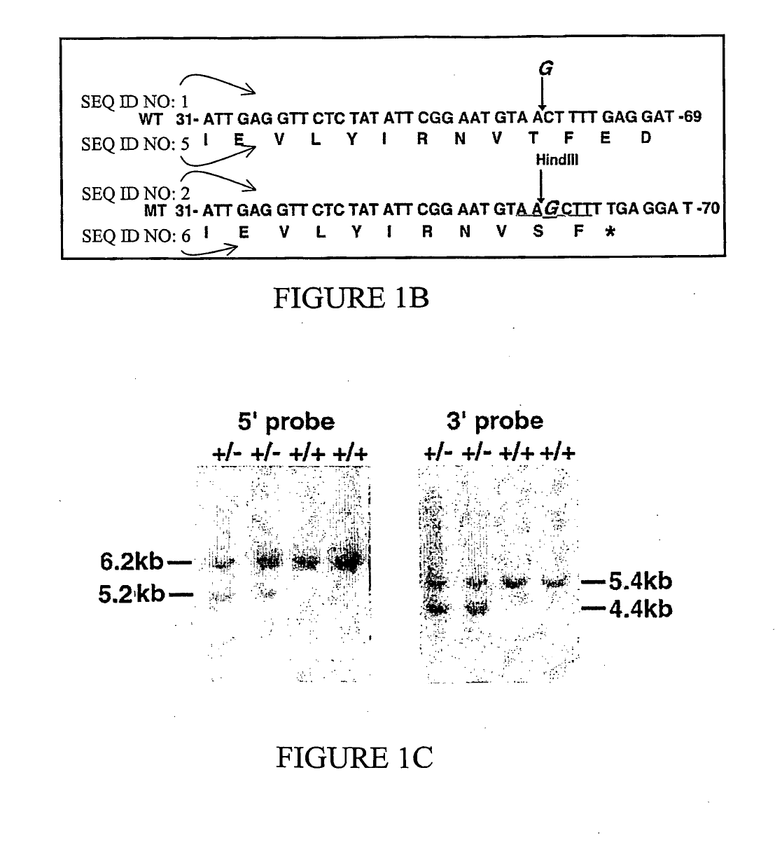

To inactivate exon 9 and thus to obtain an Fgfr2c loss-of-function mutation, the IIIc specific exon of Fgfr2c, dGTP was inserted at codon 333 (58 nucleotides after the beginning of exon 9; SEQ ID NO: 1, FIG. 1B), which created a stop codon five nucleotides downstream and produced a HindIII site 2 nucleotides upstream from the site of insertion (, NO: 2, FIG. 1B; Eswarakumar et al. 2002). Mutagenized fragments were ligated into the “Osdupdel” vector (a gift of Professor Oliver Smithies, U. North Carolina, Chapel Hill). Homologous recombination into R1 ES cells was as described previously (Arman et al., 1998).

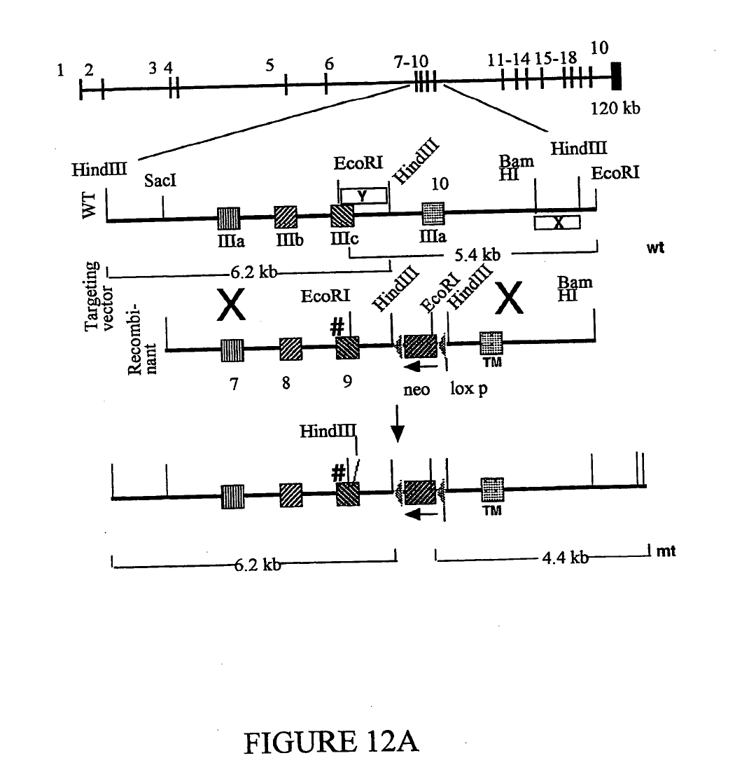

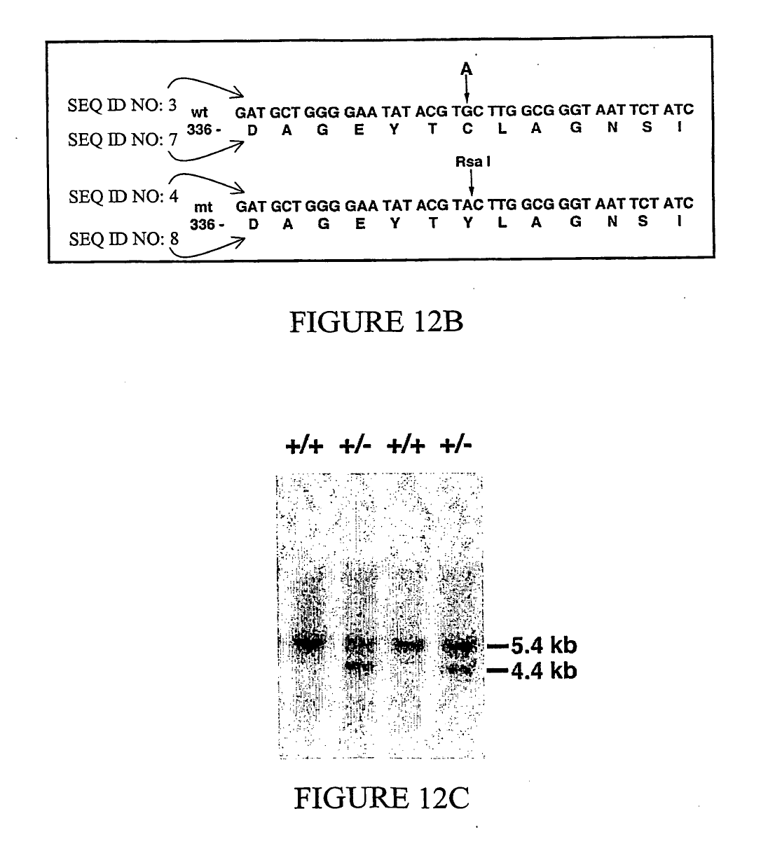

To create an Fgfr2c gain-of-function mutation Cysteine 342 was converted to Tyrosine (FIG. 12C) by site directed mutagenesis. This disrupted an intra-chain disulfide bond in the third Ig like domain of the receptor. The open half Cysteine formed inter chain disulfide bonds. These inter-chain bridges stabilized receptor dimers making the resulting dimerized receptor ligand-independent and hence constitutively active, more active than the native non-dimerized FGFR2-IIIc.

Histology and Skeletal Preparations

Cryostat or paraffin embedded sections were stained with haematoxylin-eosin. Alizarin stained skeletal preparations were made according to Kaufmnan (1992).

In Situ Hybridization

Whole mount in situ hybridization of the skull vault was according to Iseki et al. (1999). Radioactive in situ hybridization and the Fgfr2-IIIb and c probes were as described before (Orr-Urtreger et al., 19,93). The probes for Cbfa1, Ihh, PTHrP, Spp1, Collagen type II and Collagen type X are known entities.

BrdU Assay

Pregnant females were injected with 10 mg/ml BrdU (100 μg/g body weight). Embryos were post-fixed in Bouin's fixative and embedded in paraffin. Tissue sections were incubated with anti-BrdU antibody (Sigma, St. Louis) and visualized with HRP-conjugated goat anti-mouse IgG (Jackson ImmunoResearch Laboratories, PA) and peroxidase reaction. The sections were counter-stained with Mallory trichrome.

Photography

A Zeiss Axioplan, a Leitz Macroscope, or a Nikon DXM1200 microscope with a CCD camera was used.

### Example 1

### Targeted Disruption of Fgfr2c

Three different mouse strains were produced. The two experimental lines carry translational stop codons introduced by site directed mutagenesis into one of the two alternatively spliced exons, either exon 8 (IIIb) or exon 9 (IIIc) and a neomycin resistance gene was inserted into intron 9 (FIGS. 1A-B). The control line carries only the “floxed” neomycin resistance cassette in intron 9. The neo cassette was removed from the three strains by mating to an early deleter line (Lallemand et al., 1998). All three heterozygotes were normal and fertile. The control homozygote showed no phenotype, suggesting that the residual loxp site caused no defects. The Fgfr-2-IIIb^{-/-} homozygote was perinatal lethal with limb, submaxillary gland and lung agenesis.

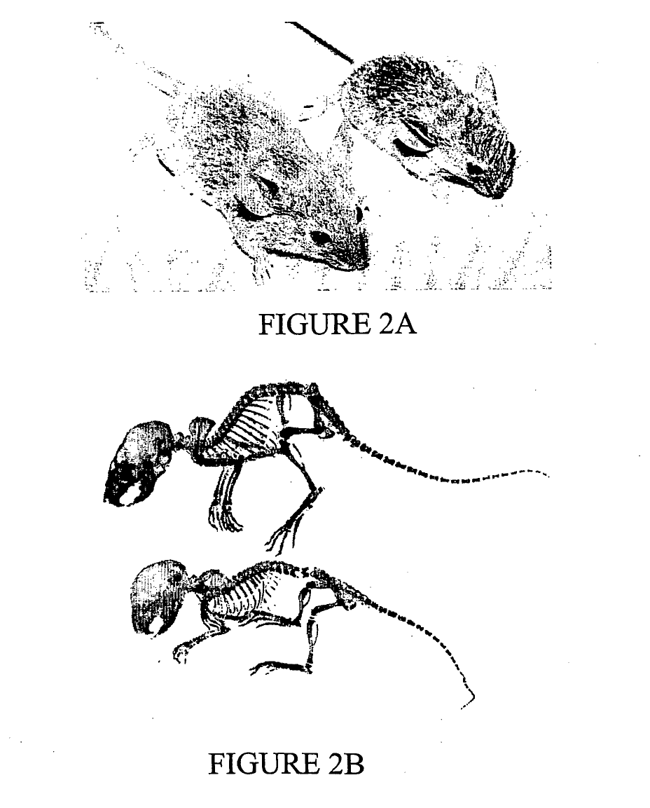

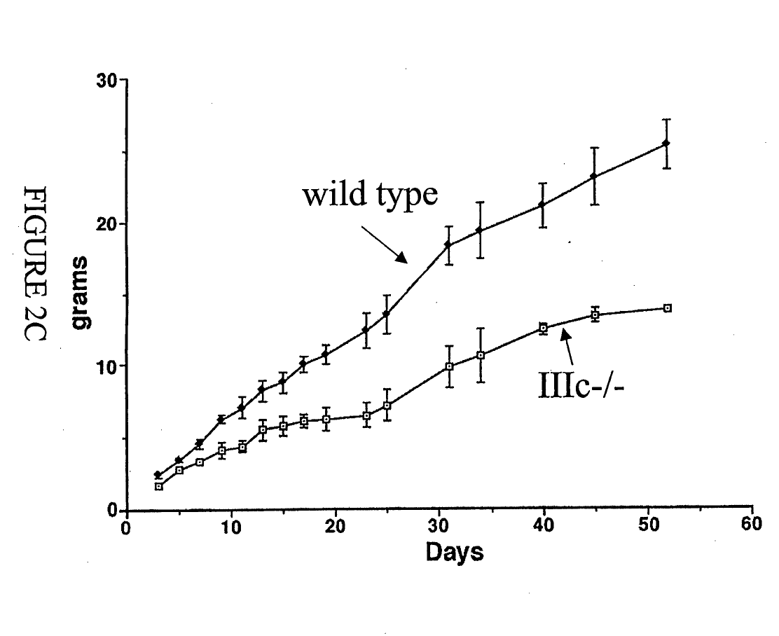

Mice homozygous for the Fgfr2c (Fgfr2-IIIc^{-/-}) mutation (SEQ ID NO: 6; Eswarakumar et al. 2002) were viable and fertile. Starting with the second postnatal week the mutant can be distinguished from its littermates by domed skull, slightly bulging eyes and shortened and sometimes bent facial area (FIG. 2A). At two weeks of age prognathia of the lower incisors develops (FIG. 2B), which in most mutants interfered with feeding; cutting back these teeth, however enabled the animals to continue to feed and develop. Some have now been alive for over a year, but stayed significantly smaller than their littermates. Skeletal preparations at 14 days post-natal reveal that although skull morphogenesis is altered, bones of the axial and appendicular skeleton retain their normal shape and proportions while remain 40-50% smaller than their wild type littermates (FIGS. 2B-2C).

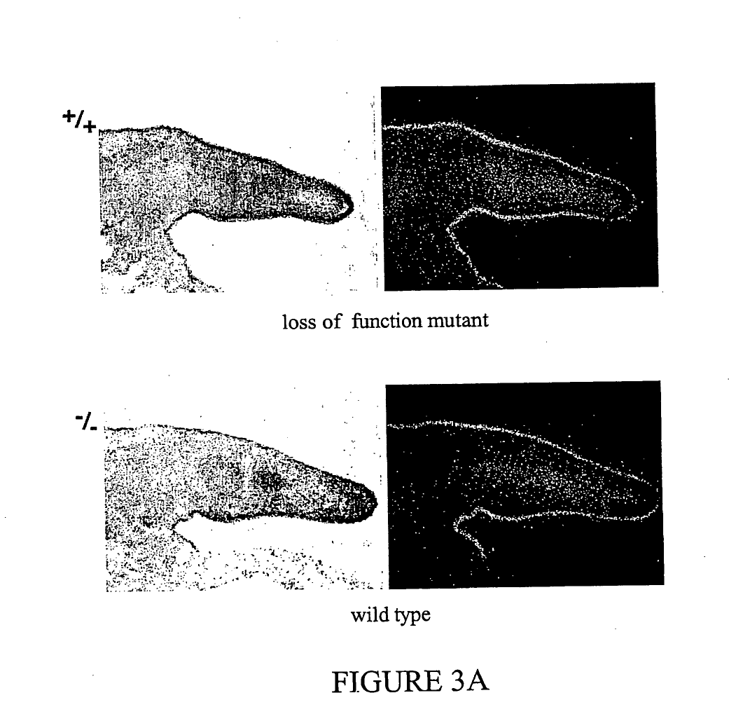

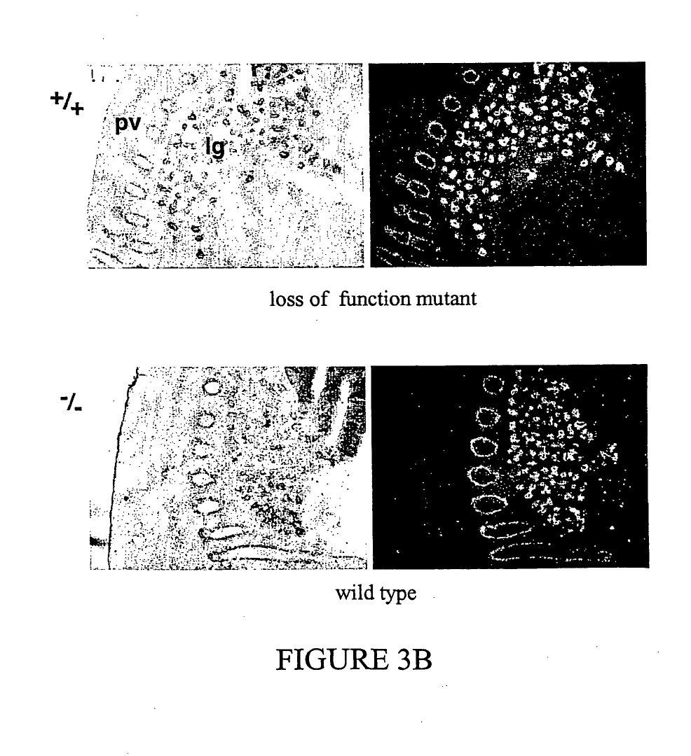

To evaluate the specificity of the Fgfr2c loss-of-function phenotype, it was important to know whether the mutation affects the expression and splicing of the alternative Fgfr2-IIIb variant. In situ hybridization of sections prepared from 12.5 embryonic-days wild type and mutant embryos, detected Fgfr2-IIIb transcripts in the surface ectoderm of the developing limb (FIG. 3A), in the perichondrium of prevertebrae (FIG. 3B, pv) and ribs as well as in the branching epithelium of the lung (FIG. 3B, lg) at 14.5 embryonic-day (FIG. 3B). This pattern of Fgfr2-IIIb expression was identical in homozygous Fgfr2-IIIb^{-/-} and wild type embryos (FIGS. 3A-3B). We therefore concluded that the point mutation in exon 9 results in a phenotype specific for the IIIc splice variant. The images indicate that characteristic Fgfr2-IIIb expression pattern in the surface ectoderm and in the bronchial epithelium of the lungs (lg) as well as in the perichondrium of pre-vertebrae (pv) is similar in Fgfr2-IIIc mutant (−/−) and its wild type littermate (+/+).

### Example 2

### Fgfr2c Expression in Developing Bone

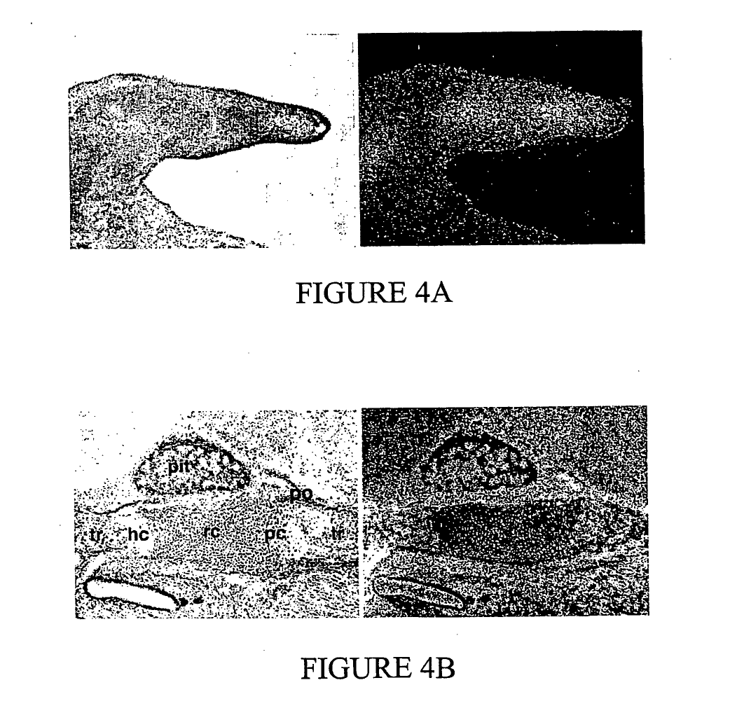

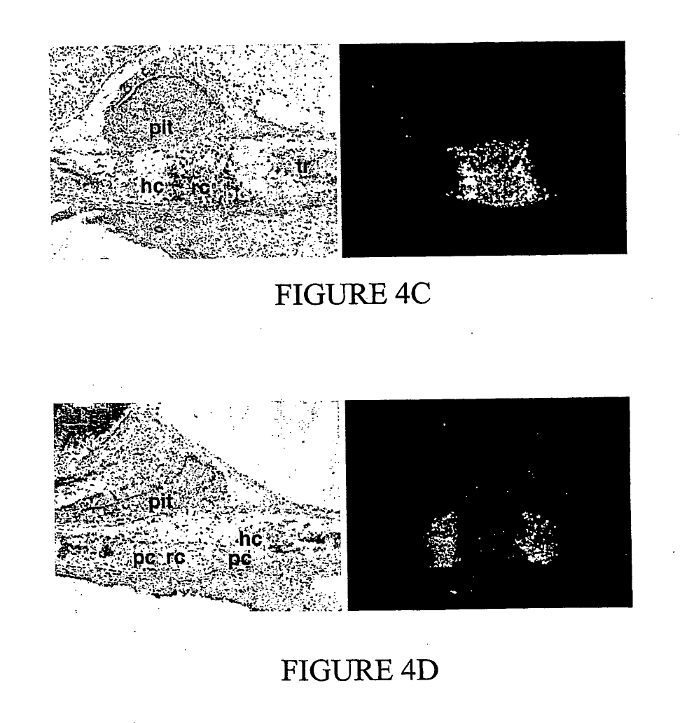

As reported before, Fgfr2c is expressed in the 14.5 embryonic-day around the cartilage models of long bones, ribs, sternum and vertebrae. Here we re-investigated the localization of Fgfr2c transcripts in the developing skeleton during primary and secondary ossification. Transcripts were detected in mesenchymal condensates of the presumptive humerus, radius and ulna (FIG. 4A). The pattern of Fgfr2c expressed in secondary ossification centers of the skull base is shown in FIGS. 4B to 4D. At the juncture of the basioccipital and basisphenoid bones, Fgfr2c transcripts were localize to the ossification zone, to the bone marrow and to the perichondrium and periosteum (po). Diffuse label was seen in the pituitary (pit), the brain and in the loose mesenchyme of the limb bud, as described previously (Orr-Urtreger et al., 1993). Specific localization of Fgfr2c transcripts in the ossification zones of the skull base was validated by the localization of type II (FIG. 4C) and type X collagen (FIG. 4D), which are respectively specific for the proliferative (pc) and hypertrophic chondrocyte (hc) layers and not for the resting chondrocyte (rc). These results indicate that the IIIc variant of Fgfr2 is first expressed in mesenchymal condensates and later in the perichondrium and the zone of ossification. The latter two localizations are characteristics for the osteoblast rather than the chondrocyte lineage.

### Example 3

### Skull Base Craniosynostosis in Fgfr2c^{-/-} Mutants

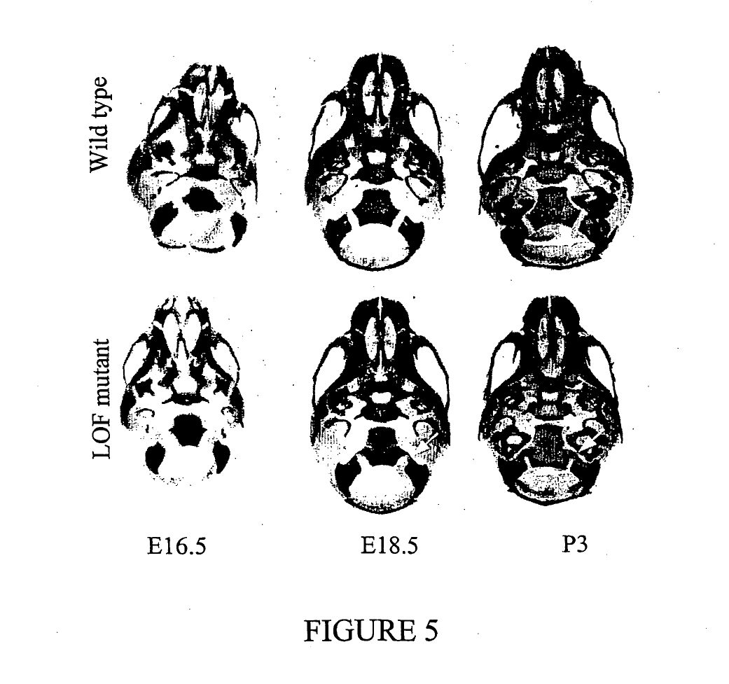

The anatomical basis of abnormal skull formation of the mutant was investigated in alizarin stained skeletal preparations. Fetal skulls between 16.5 embryonic-day to day 3 post-natal revealed reduced size of the bones in the mutant skull base (FIG. 5). By 18.5 embryonic-day the suture between the basioccipital and the exoccipital bone started to fuse (FIG. 5, arrow), and by day 3 post-natal fusion between the basioccipital and basisphenoid was also evident (FIG. 5, arrow).

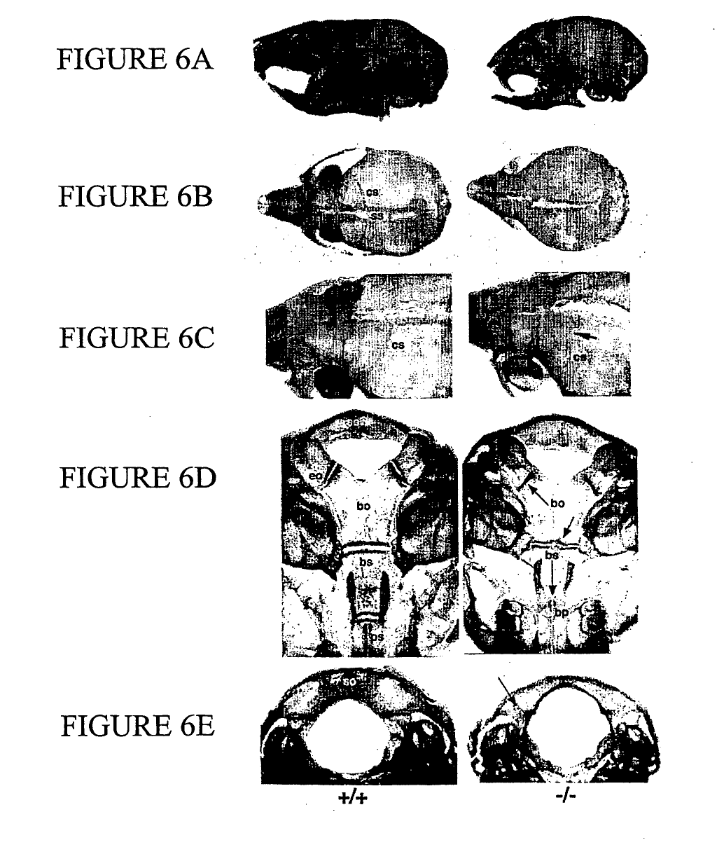

By day 14 post-natal, the mutant skull was much shorter than the wild type; both facial and neurocranial regions were affected, with down-curved nasomaxillary region, prognathia of the lower incisors and a rounded skull vault (FIG. 6A). The dermal bones of the skull vault and viscerocranium showed the same density of alizarin staining as the wild type. The metopic, sagittal (ss) and lambdoid (ls) sutures of both mutant and wild type skulls were unfused, but the mutant coronal suture (cs) showed a greater degree of overlap of the frontal and parietal bones, with partial or complete bony fusion in some specimens (FIGS. 6B-6C). Clear abnormalities were present in the skull base. Mineralization was greatly reduced, especially in the basisphenoid and orbitosphenoid (FIG. 6D). All of the skull base sutures were open in the wild type skull at this stage. In the mutant, however sutures between the basioccipital (bo) and exoccipital bones, and between the exoccipital (eo) and supraoccipital (so) bones, were fused; fusion had begun in the basioccipital/basisphenoid suture (FIGS. 6D-6E). The basisphenoid (bs)/presphenoid suture was abnormally positioned under the bony palate (bp), due to shortening of the mutant skull base (vertical arrow in FIG. 6D).

Measurement of the skull base bones revealed that most of the shortening was due to reduced growth of the middle (sphenoid) region. The wild type: mutant ratio of bone lengths on the specimen illustrated in FIG. 6D is 1:0.95 from the outer border of the supraoccipital bone to the basioccipital-sphenoid suture, but 1:0.55 from this suture to the caudal border of the maxilla. This is also evident as a reduced distance between the otic capsule and the caudal border of the tooth row on each side.

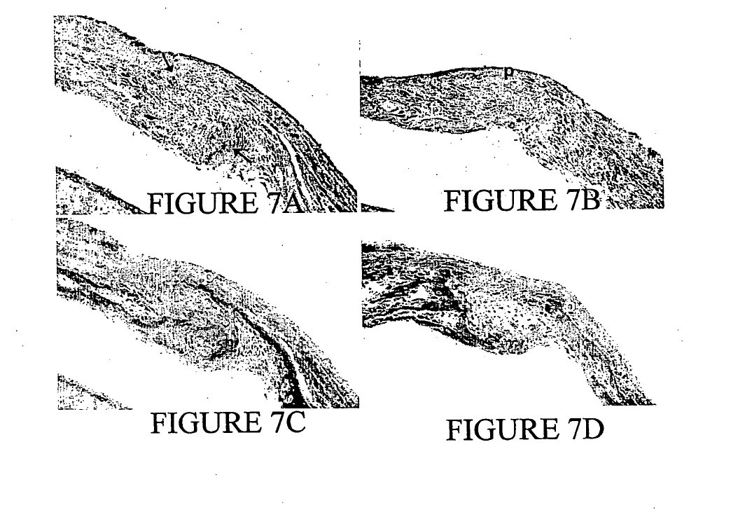

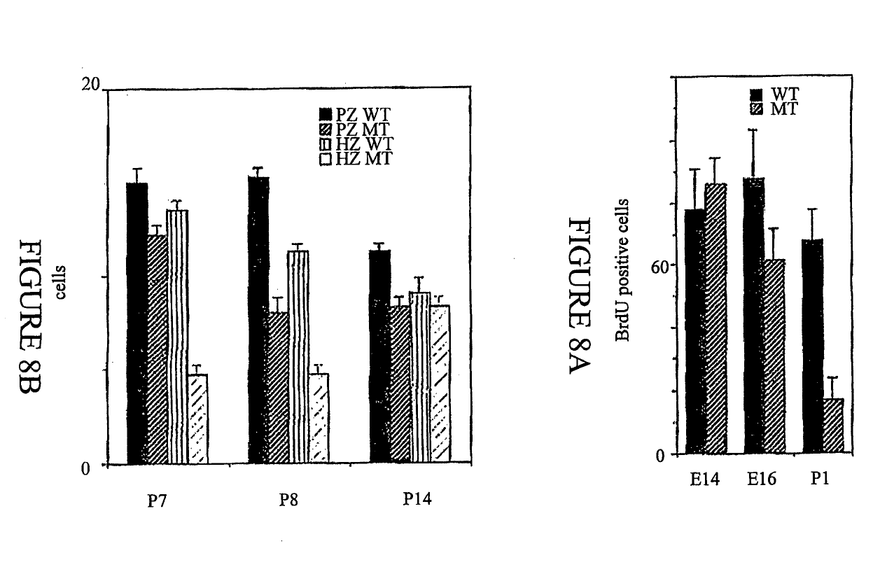

To obtain further information on the changes caused by loss of Fgfr2c, the rate of proliferation was investigated by BrdU incorporation in the region of the coronal suture, at embryonic-days 14.5 and 16.5 and day 1 post-natal (FIGS. 7A-7B and FIG. 8A). Histology (Mallory trichrome stain; FIGS. 7C-7D) showed that at all stages the sutural borders of the frontal (FIGS. 7A-D: f) and parietal (FIGS. 7A-D: p) bones were closer together, and began to overlap at an earlier stage in the mutant.

Fewer BrdU-positive cells were detected in the mutant sections at 16.5 embryonic-day and day 1 post-natal (FIG. 7B and FIG. 8A, MT) as compared to the wild type (FIG. 8A; WT). At day 1 post-natal, no BrdU-positive cells could be detected in the mutant (FIG. 7B) and the organization of the ossification fronts was lost (FIG. 7D).

Cell count in the hypertrophic chondrocyte zone (FIG. 8B; HZ) and in the proliferating chondrocyte zones (FIG. 8B; CZ), indicate lower proliferation in Fgfr2c^{-/-} mutants (FIG. 8B, MT) with respect to the wild type (FIG. 8B,WT).

Initially we assumed that the small size of the mutant was caused by starvation due to feeding difficulties because of overgrowth of the lower incisors. This now seems unlikely, since cutting these teeth back as early as possible allows the pups to survive, but growth is still reduced. Furthermore, the domed skull of mutants was already evident before the incisors erupt. We therefore suspected that the observed dwarfism was part of a general syndrome due to defective ossification.

### Example 4

### Fgfr2c is Involved in the Development of the Endochondral Skeleton

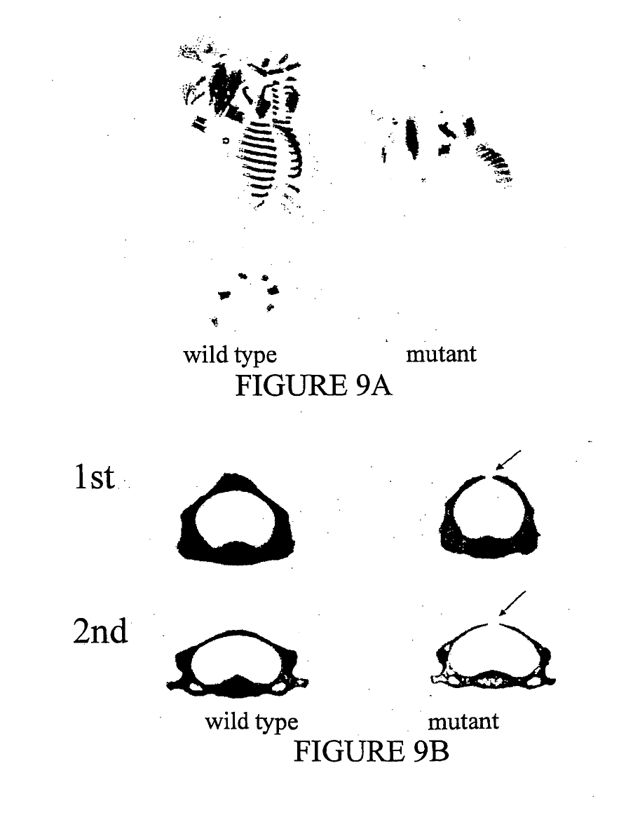

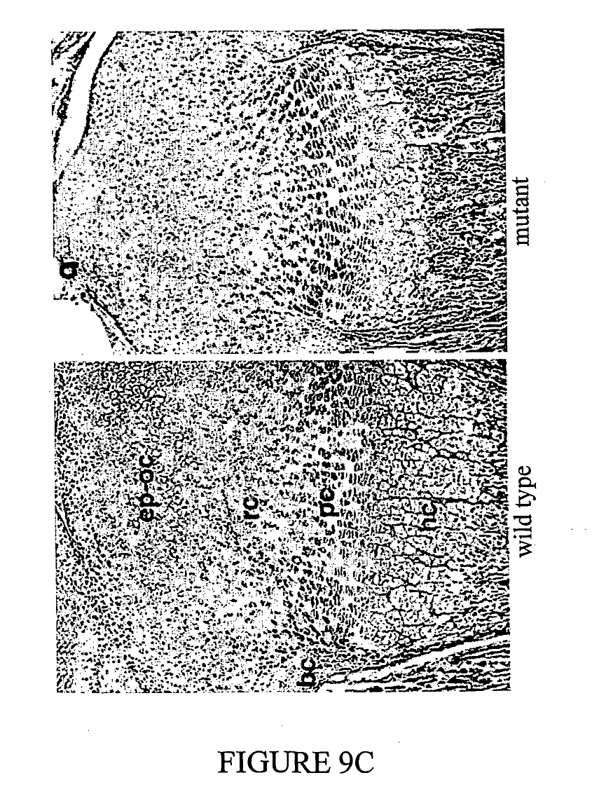

The first signs of general ossification defects were discovered in alizarin stained skeletal preparations of wild type and mutant fetuses at 14.5 embryonic-day (FIGS. 9A-9B). The onset of ossification was delayed in the mutant, so only the earliest bones to undergo ossification were stained, i.e. the mandible, clavicle, scapula (blade), humerus, and medial parts of the upper ribs (FIG. 9A). The delay affected both dermal and endochondral components of the skull vault and skull base; there was no ossification of the hindlimbs or vertebrae. Investigation of isolated vertebrae at day 14 post-natal revealed that the vertebral arches did not fuse in the mutant (FIG. 9B). It follows that Fgfr2c is required for normal mineralization of endochondral bones, hence defective osteogenesis and not inadequate feeding was responsible for dwarfism in Fgfr2c−/− mice.

As a further analysis, we investigated the tibial growth plate of 7 day-old wild type and mutant mice. FIG. 9C demonstrates a considerable narrowing of the hypertrophic chondrocyte layer with advance in the trabecular ossification front in the mutant, as compared to wild type, which was shown also by the morphometry of the proliferative and hypertrophic chondrocyte zones. This result was consistent with precocious ossification in the cranial sutures (cs) as well as in the growth plate of long bones. Whether Fgfr2c is required for the differentiation of chondrocytes or osteoblasts was investigated by in situ hybridization.

### Example 5

### Fgfr2c Affects Regulators of Osteoblast and Chondrocyte Differentiation

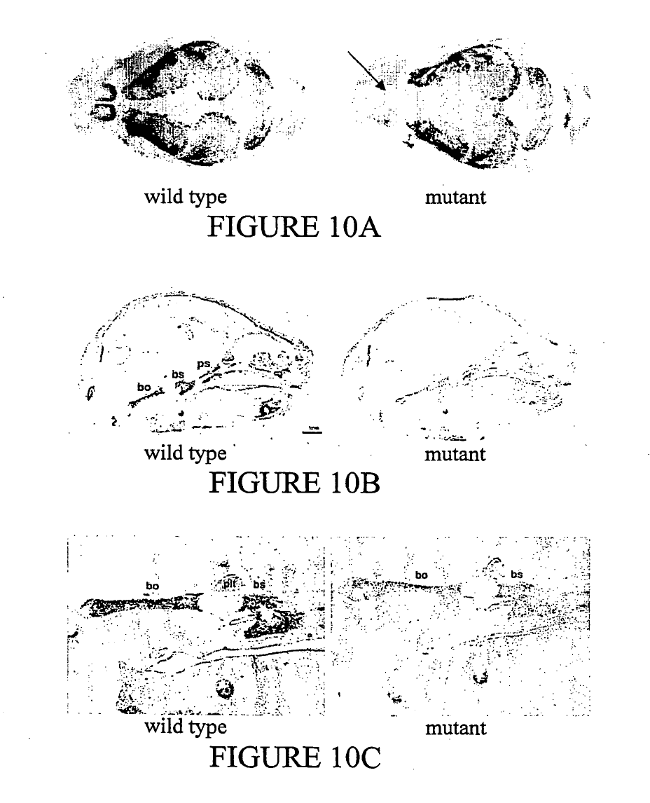

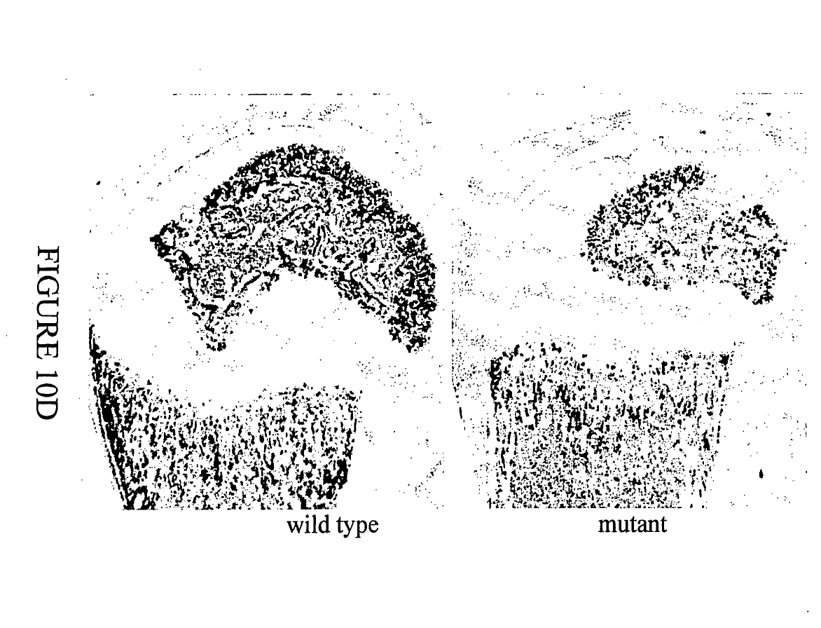

Osteopontin, which is encoded by the gene secreted phosphoprotein 1 (Spp1), is one of the major non-collagenous bone matrix proteins, produced by osteoblasts and osteoclasts. It is copiously expressed by mineralized bone and is involved in bone remodeling. Hence, Spp1 expression is a good measure of osteogenesis. Its transcripts were clearly distinguishable at 18.5 embryonic day in the skull vault. Whole mount in situ hybridization demonstrated considerable decrease in the level of Spp1 transcripts in the mutant fronto-nasal and frontal bones (FIG. 10A). They were also obvious in the wild type skull base (FIG. 10B). Spp1 expression in all skull bones of the mutant was considerably lower than in the wild type (FIG. 10C). Investigating the tibial growth plate at day 15 post-natal revealed a similar decrease of Spp1 transcription in the epiphyseal plate and in the mineralizing periphery of the secondary ossification center of the tibial head (FIG. 10D). These observations indicate that Fgfr2c promotes Spp1 expression in the dermal bones of the skull, as well as in the endochondral bones of the skull base, occiput and appendicular skeleton.

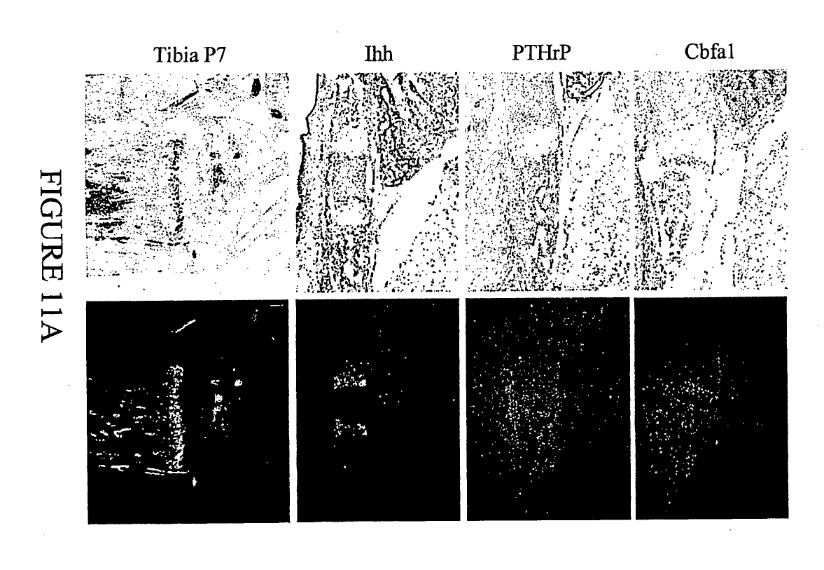

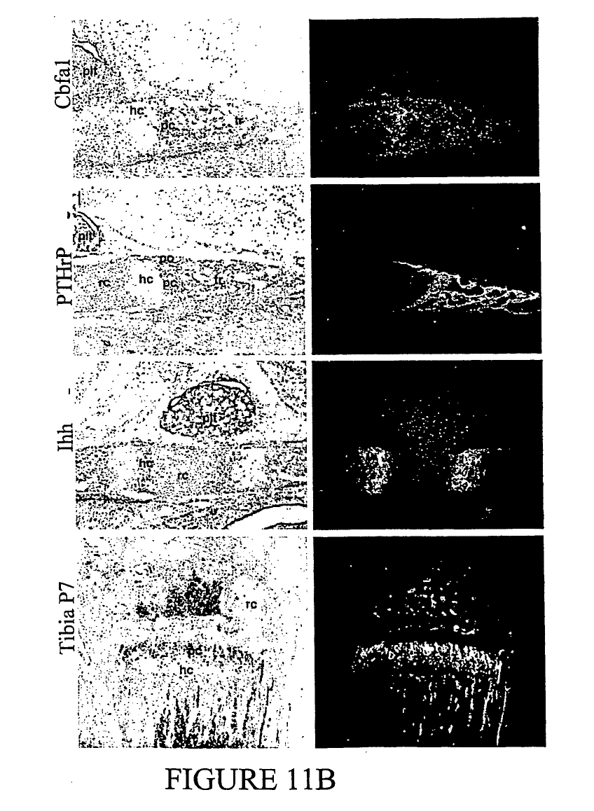

To investigate further the effect of Fgfr2c in the osteoblast lineage Cbfa1 expression was investigated in the skull base on sagittal sections of fetuses at 18.5 embryonic-day (FIGS. 11A). Cbfa1 was expressed in the ossification zone, in and around the bony trabeculae of the basioccipital bone. In the mutant however this label was much reduced (FIG. 11A) with respect to the wild type (FIG. 11B). It follows that Fgfr2c signaling is required for the normal expression of both Cbfa1, which is detectable in early osteoblasts and Spp1, which is expressed in mature osteocytes during mineralization.

During endochondral bone formation and growth, chondrocyte and osteocyte precursors migrate into mesenchymal condensates, which form at sites of endochondral ossification. Proliferating chondrocytes in the bone shaft and later in the growth plates undergo hypertrophy and finally apoptosis. They produce a cartilage framework, which is ossified due to mineralization of matrix proteins deposited by invading osteoblasts. Chondrocyte differentiation is orchestrated by a reciprocal regulatory loop between Indian hedgehog (Ihh) and the parathyroid hormone-related peptide (PTHrP). We investigated the effect of the Fgfr2c mutation on PTHrP and Ihh expression (FIG. 11). Expression of PTHrP in the perichondrium, osteogenic front and resting chondrocyte zone of the skull base of the mutant was suppressed (FIG. 11A) with respect to the wild type (FIG. 11B). Accumulation of Ihh transcripts in the pre-hypertrophic and hypertrophic chondrocyte layer of the skull base and tibial growth plate was also reduced (FIG. 11A), albeit to smaller extent as compared to the wild type (FIG. 11B). Taken together these data, show that loss of Fgfr2c affects both chondrocyte and osteocyte differentiation.

### Example 6

### Targeted Gain of Function Mutation of Fgfr2c

The Fgfr2c gain-of-function mutation displays the expected phenotype, namely, induction of bone growth and up-regulation of transcription of bone regulatory genes, both of the osteoblast lineage (osteopontin and Cbfa-1) and of the chondrocyte lineage (PTHrP and Ihh), confirming that Fgfr-2c is indeed a positive regulator of osteogenesis.

In contrast, loss of FGFR3 function results in the overgrowth of long bones and the up-regulation of genes involved in osteogenesis, whereas its gain of function mutation causes achondroplasia in man and serious down-regulation of osteogenic factors in vitro.

The codon encoding Cysteine (FIGS. 12A-B, SEQ ID NO: 7; Orr-Urtreger et al., 1993) in exon 9, which controls the 3′ portion of the third Ig-like loop of Fgfr2 was mutated (FIG. 12A). Substituting dATP instead of dGTP at position 342 changed the TGC Cysteine codon into the TAC, Tyrosine codon and new restriction enzyme polymorphisms and a new RsaI site were created (SEQ ID NO: 4, FIG. 12A-B; Reardon et al., 1995). Finally a neomycin resistance cassette was inserted into intron 9. After identifying the recombinant ES cell clones with the 3′ external probe (FIG. 12C), they were further analyzed for recombination at the 5′, using an internal probe (Y in FIG. 12A) after HindIII digestion. The recombinant clones gave the expected single 6.2 kb fragment, indicating correct homologous recombination and demonstrating that there was no random multiple integration in the genome. Homologous recombinant ES cells were aggregated with four-cell embryos of the MF1 non-inbred mouse strain and the resulting chimeras were mated to the early Pgk-Cre^{m} “deleter” strain (Lallemand et al., 1998) to remove the neo cassette. The ensuing Fgfr2C^{42Y} mutant strain was maintained by back crossing to MF1.

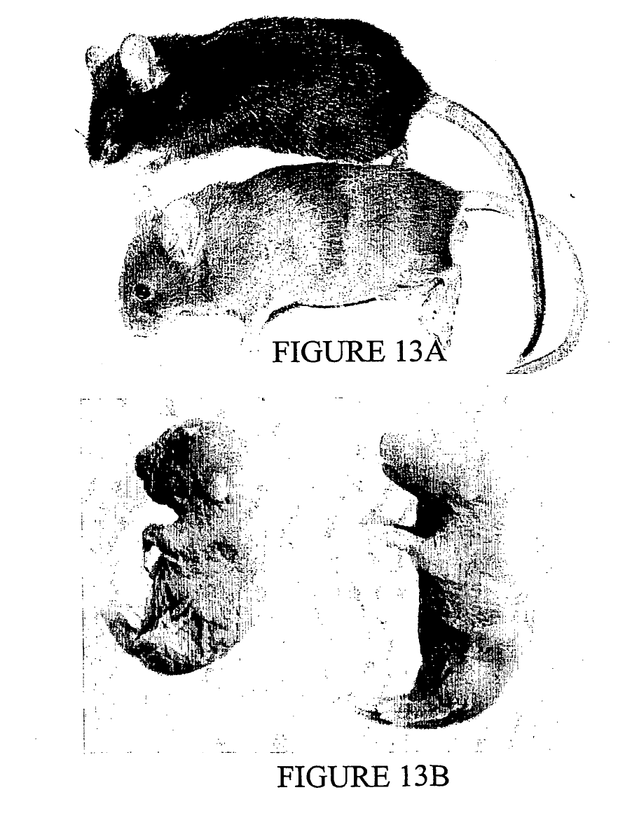

Fgfr2c^{C342Y/+} heterozygotes were viable, reached normal size and had unimpaired fertility. They were characterized by shortened face and domed skull with slightly bulging eyes (FIG. 13A bottom). Some heterozygotes had a lateral deviation of the nasal area, causing incomplete closure of the incisors that led to difficulties in feeding. In contrast to heterozygotes, all homozygous Fgfr2c^{C342Y/C342Y} mutants died due to respiratory failure immediately after birth and their stomach contained no milk. The homozygotes were 10-20% smaller than their wild type littermates and were distinguished by extremely shortened facial area and open eyelids (FIG. 13B left).

### Example 7

### Skull Vault Craniosynostosis in the C342Y Heterozygote

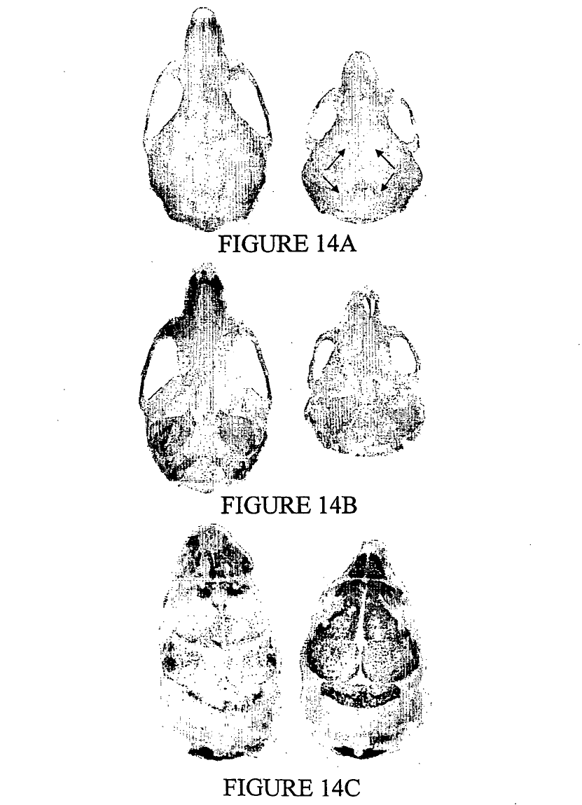

Skull morphogenesis was investigated using alizarin stained skeletal preparations (FIGS. 14A-14B). The skull of Fgfr2c^{C342Y/+} mice was rounded due to shortening of the rostro-caudal axis (FIG. 14A, right). On the basis of manipulating with forceps the coronal sutures were completely fused and the lambdoid suture partially fused, while the sagittal suture, which does not close in the mouse, remained partially separable (FIG. 14A). In the lambdoid and sagittal sutures small bone inserts (Wormian bones), could be seen and the occipital bone was fenestrated. The relative size of the parietal bone, compared to wild type increased, whereas the frontal and nasal bones were considerably smaller, resulting in a grossly shortened skull with a lateral naso-maxillary deviation in some animals. The neurocranium was round and the skull vault domed. The skull base was also shortened and the third molar was missing (FIG. 14B). In contrast to the loss of function mutation of Fgfr2c, where the basioccipital-exoccipital and basioccipital-basisphenoid sutures were precociously fused (FIG. 5), in Fgfr2c^{C342Y/+} mice, these sutures, remained open as in age matched wild type skulls (FIG. 14B).

No significant change was observed in the axial and appendicular skeleton of the heterozygote. Obvious mutant defects were fusion of the coronal and lambdoid sutures of the skull vault with bulging eyes and shortened and sometimes asymmetric skull (FIG. 14A). It follows that in contrast to the Fgfr2c loss of function mutation (FIG. 6), in C342Y heterozygotes craniosynostosis of the skull vault dominates. Skull vault craniosynostosis, shallow orbits and ocular proptosis with no obvious limb defects are hallmarks of the human Crouzon syndrome.

Evidence for gain of Fgfr2c function of the C342Y mutation was obtained by investigating osteopontin expression in the skull vault. Osteopontin, encoded by the gene, secreted phosphoprotein 1 (Spp1), is one of the major non-collagenous bone matrix proteins, produced by osteoblasts and osteoclasts. It is copiously expressed by mineralized bone and is involved in bone remodeling. Whole mount in situ hybridization revealed significant increase in Spp1 expression in the nasal, frontal, parietal and occipital bones (FIG. 14C, right), as compared to wild type (FIG. 14C, left). As expected from a gain of function mutation this increase of transcription was opposite to what was observed with the loss of function mutation of Fgfr2c, which displayed a significant decrease of Ssp1 expression in the skull vault. Since osteopontin contributes to the bone matrix, this result reinforced our conclusion that Fgfr2c contributes to functions of the osteoblast lineage.

### Example 8

### Cleft Palate, Multiple Synostosis and Lung Defects in the C342Y Homozygote

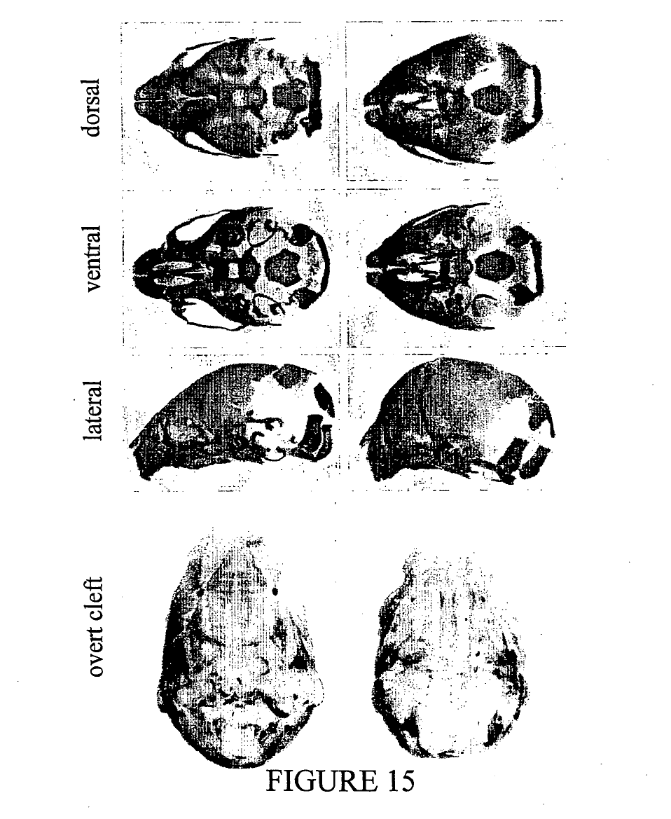

The skull of newborn Fgfr2c^{C342Y/C342Y} mice was extremely shortened in the naso-maxillary region with domed and round cranium (FIG. 15, right) as compared to the wild type (FIG. 15, left). The ossification front of the parietal, frontal and occipital bones was serrated and pitted with small fenestrae. Despite these signs of accelerated osteogenesis, the cranial sutures were more open in the mutant at this stage than in the wild type. As shown in FIG. 15, the secondary bony palate of the mutant failed to close. Overt cleft palate (FIG. 15) was observed in three of four homozygotes, in one of fifteen heterozygotes, but in none of seven homozygous wild type offspring from three Fgfr2c^{C342Y/+} F1 crosses. Meckel's cartilage, including the proximal malleus/incus region was thicker in homozygote than in wild type or heterozygous pups, and the developing malleus/incus ossicular joint (detectable in wild type skeletons) could not be detected by examination under the dissecting microscope, suggesting conductive deafness. The tympanic bone was more massive but less well ossified distally in homozygous than in wild type pups (FIG. 15).

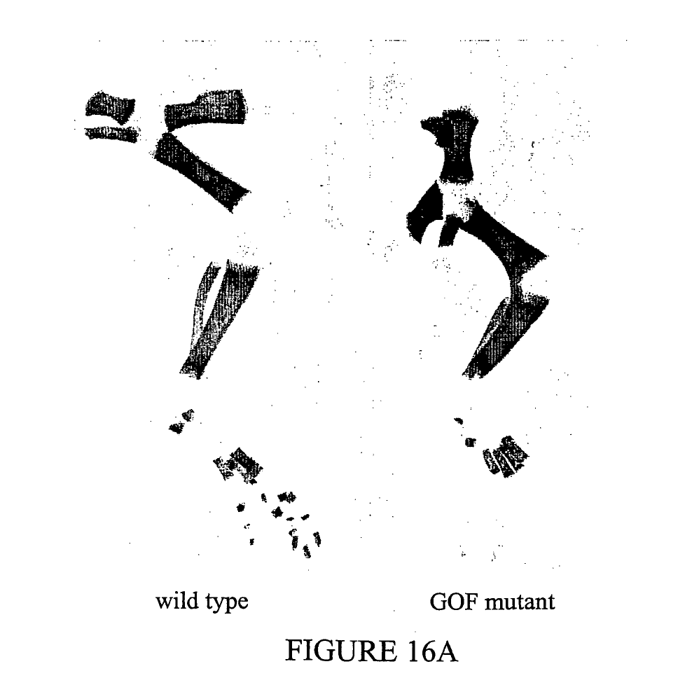

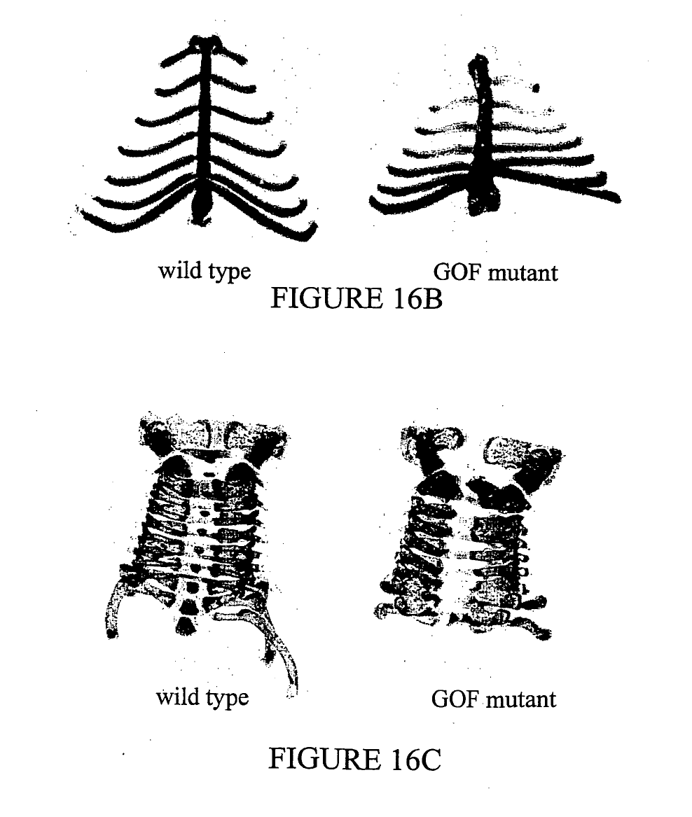

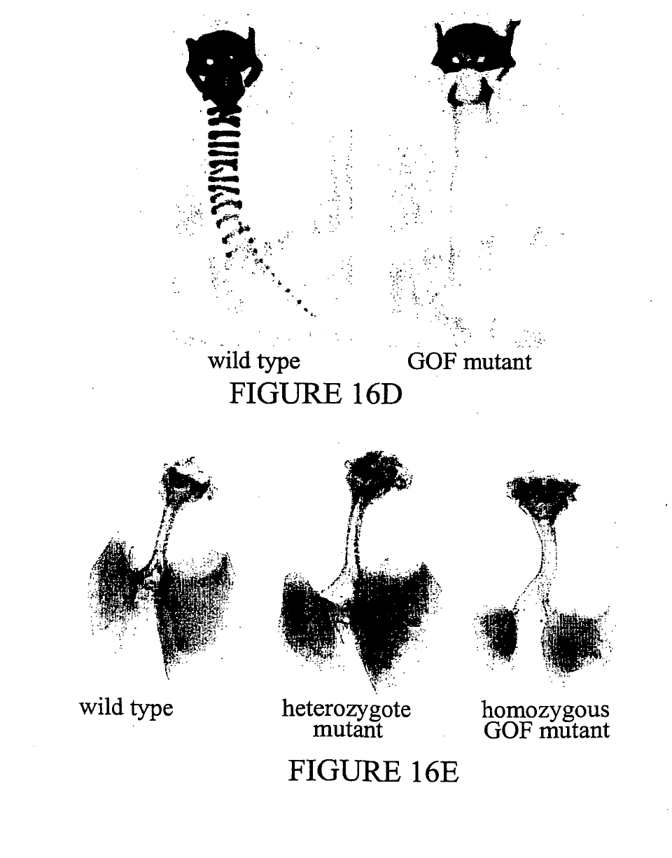

Signs of accelerated osteogenesis could be observed in alizarin stained skeletal preparations, which reveal mineralized bone. The long bones of E18.5 and newborn mutants were more robust than in the wild type and in most individual the knee joint was fused by a posterior bony bridge (FIG. 16A). A quantitative measure of increased bone density was obtained by measuring average detention units (ADU) of the X-ray radiograms along the length of the tibia using the computer programs of the Faxitron X-ray unit. Table 1 demonstrates decrease of photon capture already in the heterozygote and more significantly in the homozygote as compared to wild type. Synostosis of the stemebrae in the robust and somewhat bent sternum and fusion of the lower cervical and upper thoracic, as well as sacral and caudal vertebrae complete this picture of enhanced ossification (FIGS. 16B-D).

TABLE 1
Increase of bone density in the C342Y mutation^{#}.

AVERAGE DETENTION UNITS (ADU)*

| | Mean ± S.D. | Maximum-minimum\hat{ } |
|---|---|---|
| Fgfr2c^{+/+} | 699 ± 13.5 | 727-657 |
| | 721 ± 22.8 | 772-667 |
| Fgfr2c^{C342Y/+} | 682 ± 16.1 | 720-648 |
| | 685 ± 16.7 | 721-650 |
| Fgfr2c^{C342Y/C342Y} | 640 ± 14.0 | 660-580 |
| | 647 ± 9.3 | 669-626 |

^{#}Calculation of average detention units from X-ray radiograms of two experiments.
*ADU - a measure of photons reaching the detector across the tissue. Higher density will result lower figures. Average of 110-150 pixels were measured along the same distance in the length of the tibia.
\hat{ }the highest and lowest values encountered among the 110-150 pixels measured

An unexpected observation was the asymmetry of the rib cage, with the second rib missing and an eighth rib added at the left side (FIG. 16B) and absence of the body of cervical vertebrae (FIG. 16C). Defective development of the tracheal cartilage rings fused into a thin cartilaginous sleeve (FIG. 16D) was associated with underdeveloped lobes of the lung (FIG. 16E). These tracheal and lung defects were present in most homozygotes. They and the frequently observed cleft palate may explain the lethality of this phenotype.

### Example 9

### Gain of Fgfr2c Function Results in Increased Cbfa1/Runx2 Expression

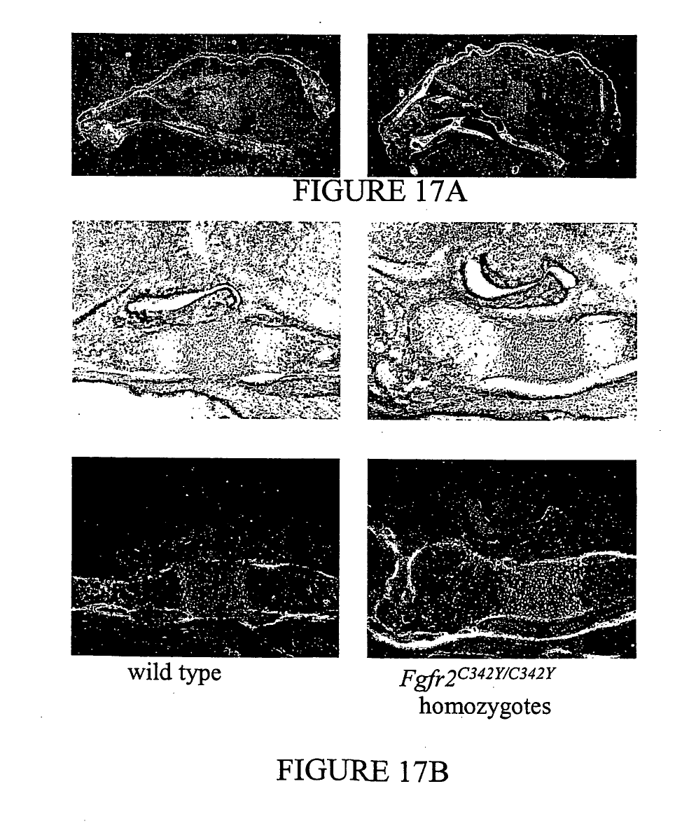

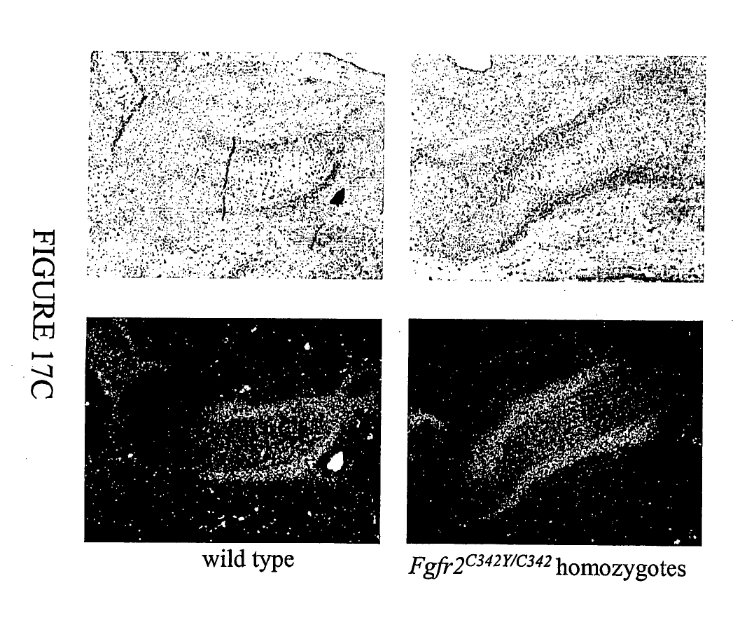

Cbfa1/Runx2 is a master gene of the osteocyte lineage and when inactivated only the chondroskeleton is formed, although it has certain effects on the chondrocyte lineage as well. We investigated Cbfa1 expression in Fgfr2c^{C342Y/C342Y} newborn mice. In situ hybridization of sagittal sections of the skull revealed robustly thickened nasal and palatal bones and thickened and curved skull base, coupled with increased expression of Cbfa1 in the bones of the skull base, skull vault and naso-maxillary area (FIGS. 17A and 17B). Higher magnification of the basioccipital-basisphenoid junction revealed that the Cbfa1 transcripts accumulate in the bone collar, periosteum and perichondrium and present heavier signals (extending throughout the perichondrium) in the mutant than in the wild type (FIGS. 17C-17F). The E13.5 humerus was investigated as an example of early osteogenesis: Cbfa1 expression was stronger in the periostelirn and proliferating chondrocyte layer of the mutant than the wild type (FIGS. 17G-17J). Since enhanced osteopontin expression in the C342Y heterozygote and enhanced Cbfa1 expression in the homozygote gain of function phenotype were the opposite to our observations in the loss of function mutation (FIG. 11), these results confirm that Fgfr2c exerts positive control on these genes.

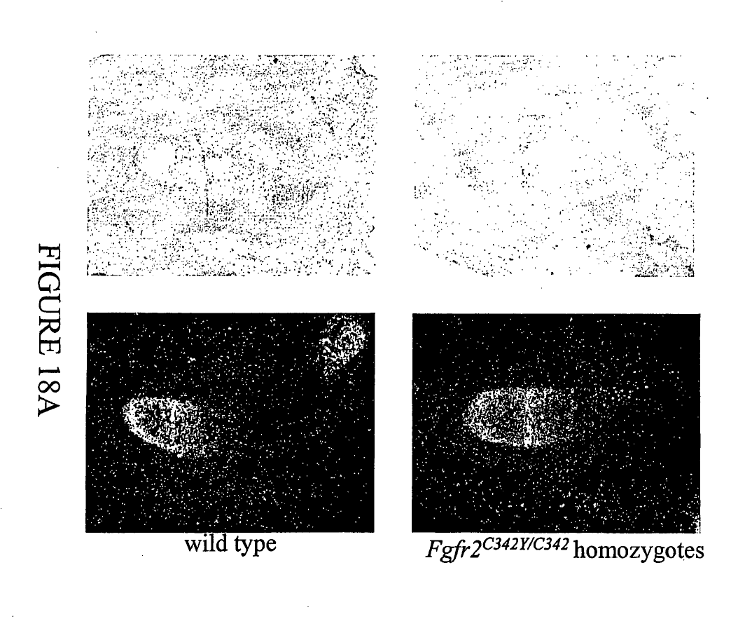

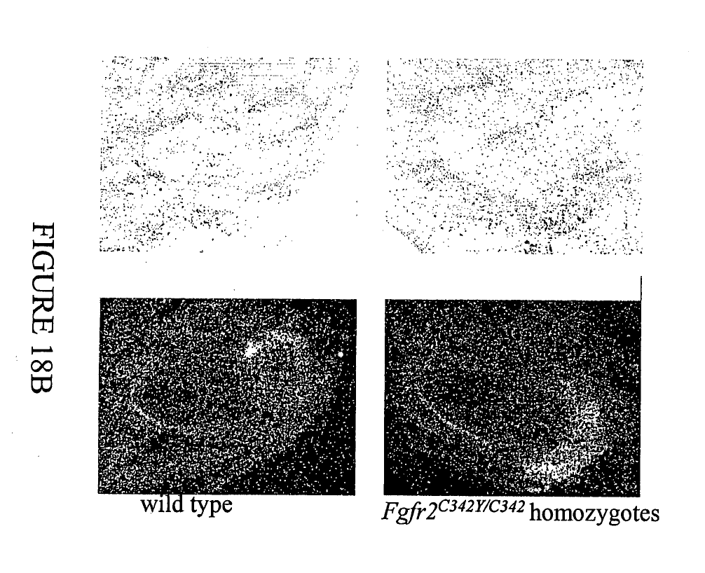

Chondrocyte differentiation is orchestrated by a regulatory loop between Indian hedgehog (Ihh) and the parathyroid hormone-related peptide (PTHrP). Recent data emphasize that Ihh, besides its involvement with chondrocyte differentiation, is also a mediator of chondrocyte proliferation. We investigated the effect of the Fgfr2c gain of function mutation on Ihh and PYHrP expression in E13.5 embryos, during early stages of ossification. Ihh transcription was detected in the perichondrium as well as in the pre-hypertrophic center of the late cartilage model of the E13.5 humerus; the expression domain of the mutant was increased compared to wild type, but its level was unaffected (FIGS. 18A-18D). PTHrP was expressed in the perichondrium and in pre-hypertrophic chondrocytes of the prospective epiphysis. The area of expression in the mutant epiphysis was greater than in the wild type, but its level did not seem to be altered (FIGS. 18E-18H).

It follows from these results that with enhanced osteogenesis the area of chondrogenesis also increased in the homozygous mutant, but no significant increase was observed in the level of Ihh and PTHrP expression. It was however still possible, without wishing to be bound by any particular theory, that Fgfr2c acts at an earlier stage of chondrogenesis. Sox 9, which regulates expression of the chondrocyte specific Col2a gene encoding type II collagen, is expressed in all cartilage primordia. In situ hybridization with Sox9, Col2a and another cartilage specific collagen gene, Col10a, did not however reveal a difference between wild type and newborn Fgfr2c^{C342Y/C342Y} mice.

Taking these data together, enhanced function of Fgfr-2c increases the expression of Ssp1 and Cbfa1, without greatly influencing that of Ihh, PTHrP and other genes involved with the chondrocyte lineage. This distinguishes the gain of function and loss of function mutations of Fgfr2c. While in the loss of function mutation retarded osteogenesis was associated with a significant decrease of Ihh and PTHrP transcription (FIG. 11), in the gain of function mutation, transcription of the chondrocyte specific genes did not change significantly in comparison to the expression of genes associated with osteocyte function.

### Example 10

### Fgr2c Gain-of-function Mutation is Beneficial for Fracture Healing

The experimental set up for the bone fracture experiments included a calibrated weight, which was allowed to fall from an experimentally established height on the tibia of mice in deep narcosis. Before applying the weight, a titanium pin was introduced into the marrow cavity of the tibia to stabilize the bone to be fractured. For the first three days following such procedure mice were constantly treated with painkillers.

Each experimental group consisted of heterozygous Fgfr2^{gof/+} mice and their homozygous wild type littermates.

X-ray Imaging

Fracture healing was weakly monitored by applying X-ray imaging. At the end of each week, ending with the fifth week after fracture, animals were sacrificed for histological analysis. This is a minimal model, because the heterozygote is completely normal, save shortening of the craniofacial area and skull vault craniosynostosis. In contrast to the heterozygote, the homozygote displays extensive synarthroses with increased bone density in the entire skeleton and dieing at birth with cleft palate, trachea and lung defects.

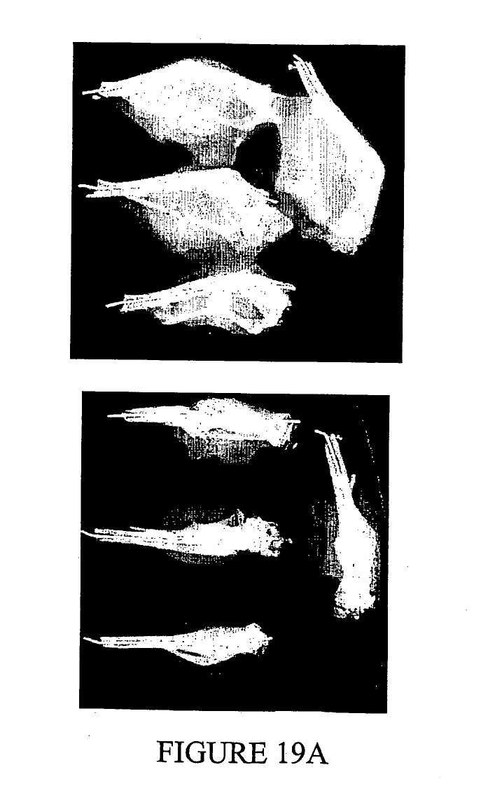

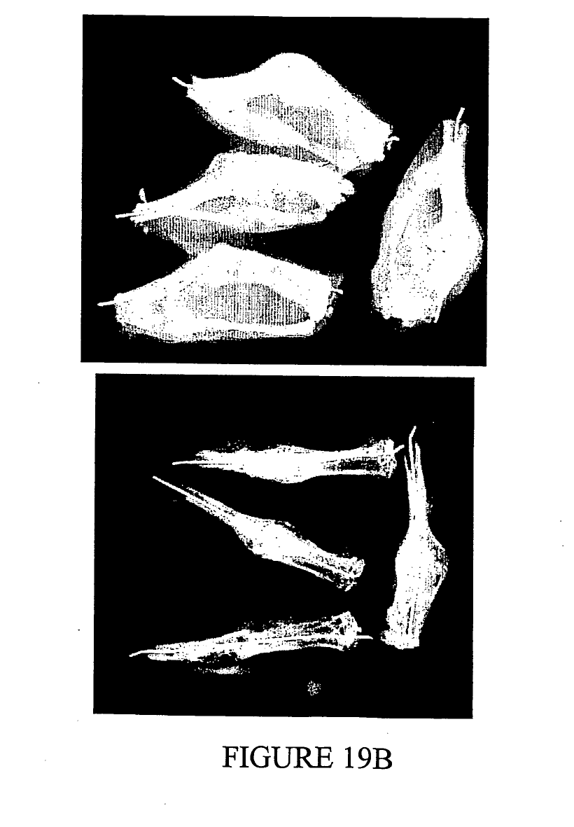

The X-ray images indicated that the heterozygous mutant group and control (wild type) group could be distinguished already at the first week after the fracture procedure by considerably smaller callus than that of the wild type. This became especially obvious by the third week following the fracture procedure, when the fracture line became invisible in the tibiae of three out of four mutant mice (FIG. 19A) and in one of the four control mice (FIG. 19B).

Histology—H&E Staining

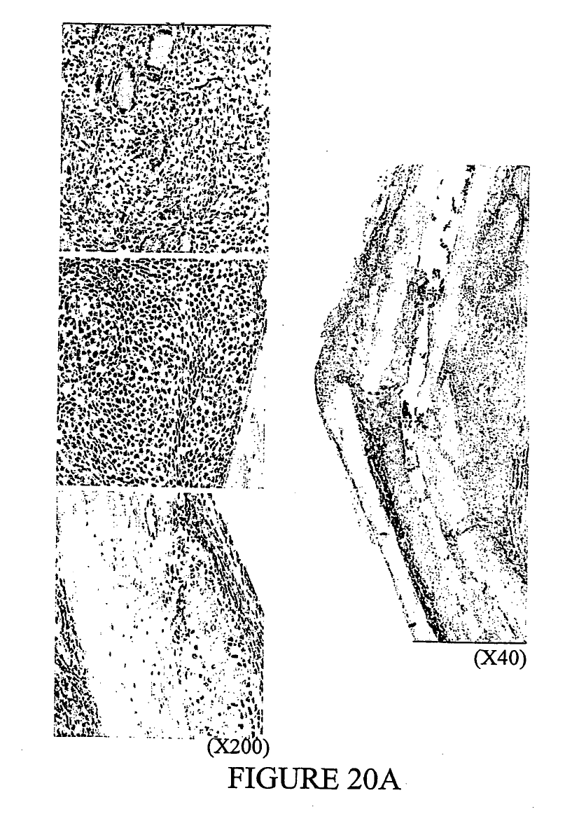

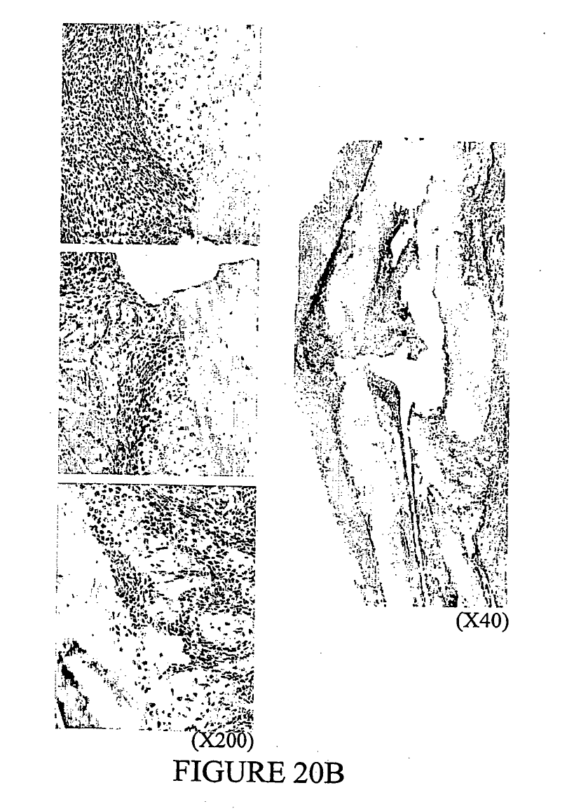

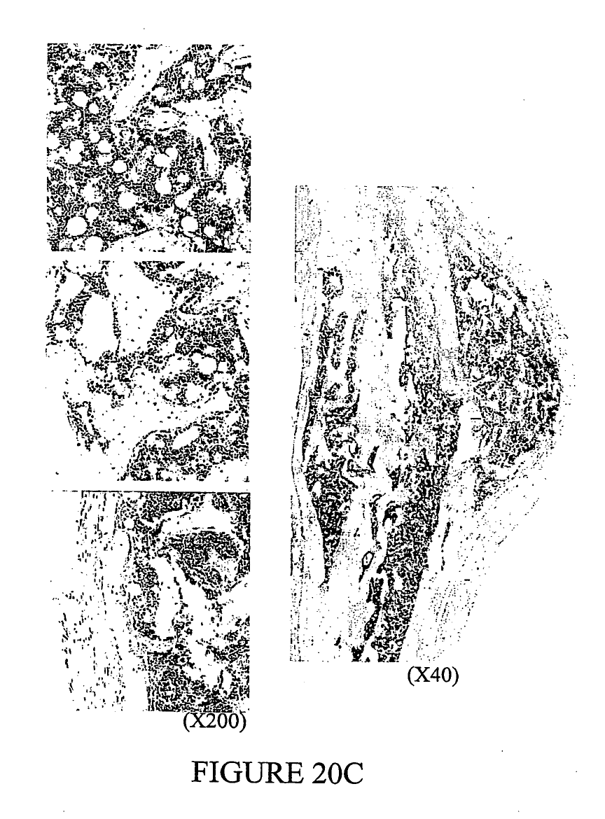

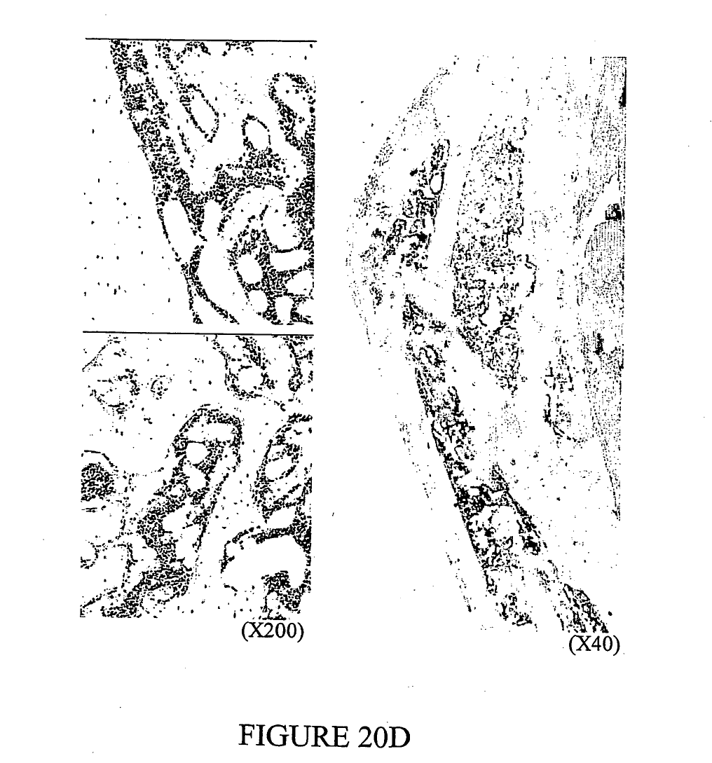

One week after the fracture procedure a large fibro-cartilageneous callus with heamorage and necrosis in the fracture line alone with a peri-osteal and peri-trabecular bone formation was observed in the control group (FIG. 20A). In the mutant the callus was smaller and its structure more compact (FIG. 20B).

Five weeks after the fracture procedure a bony union was seen in the control group, which had a large bony callus with trabecular bone formation, while in the mutant group the fusion was accompanied by a much more compact and smaller callus, with less trabecular and more cortical bone formation (FIG. 20C). Moreover the periosteal reaction of the mutant was much more compact, narrow and solid (FIG. 20D).

The above data indicates that fracture healing progresses faster in the gain of function mutant than in wild type control. In fact, the mutant was about one week ahead of the wild type, as evaluated by:
a. The amount of cartilage in the callus, which was much less in the mutant than that in the wild type.
b. Consolidation of the periosteal reaction that was more advanced in the mutant.
c. A bony union, which was incomplete in the wild type group, while was present in all animals of the mutant group at three weeks after fracture.

### Example 11

### Molecular Evidence for Enhanced Fracture-related Ossification in Fgfr2c Gain-of-function Mutation

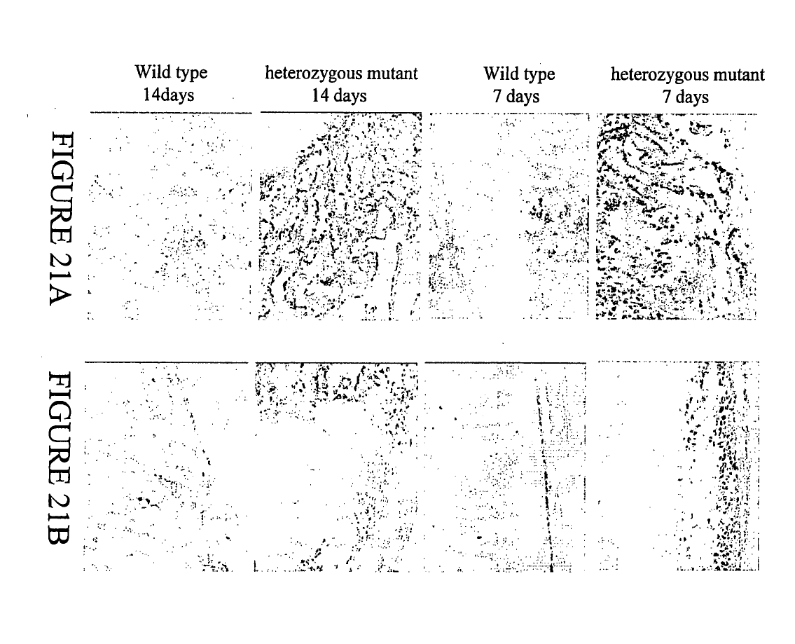

Osteopontin and osteocalcin are extracellular matrix proteins produced by osteoblasts and osteoclasts. Non radioactive in-situ hybridization with these two markers at the site of experimental fraction, osteopontin (FIG. 21A) and osteocalcin (FIG. 22B), revealed increased transcription in the gain of function heterozygous mutant compared to wild type control exhibit increased osteopontin transcription. With both markers, an amplified hybridization was observed in the more extensive trabecular bone of the mutant.

This finding suggests more extensive osteoblast activity in the mutant and since actual ossification takes place through calcification of the bone matrix, these results provide a molecular evidence for enhanced fracture-related ossification due to gain of Fgfr2c function.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### REFERENCES

Arman, E., et al. (1998) Proc. Natl. Acad. Sci. USA 95,5082-5087.
Fang et al. (1997) Anal Biochem 254, 139-143.
Eswarakumar et al., (2002) Development 129, 37853.
Griscelli, et al. (1998) Hum Gene Ther, 1919-1928.
Kaufman, M. (1992) Academic Press.
Lallemand, Y. (1998) Transgenic Res 7, 105.
Langner, et al. (1998) Adv Exp Med Biol 451, 415.
Mangasarian et al., (1997) J Cell Physiol., 172 117.
Ornitz, D. M., et al. (1996) J. Biol. Chem. 271, 15292.
Orr-Urtreger, A., et al. (1993) Developmental Biol 158, 475-486.
Pillai, et al. (2001) Curr Opin Chem Biol 5, 447.
Reardon W. et al., (1994) Nat Genet. 8, 98.
Sambrook, J., et al. (1989) 2nd ed., Cold spring laboratory press, Cold spring harbor, N.Y., USA.

1. A polynucleotide construct comprising a polynucleotide sequence encoding an active variant of FGFR2c and a polynucleotide sequence encoding at least one FGFR2c specific ligand.

2. The polynucleotide construct of claim 1, comprising a promoter operatively linked to the polynucleotide sequence encoding FGFR2c.

3. The polynucleotide construct of claim 3, wherein the promoter is tissue specific.

4. The polynucleotide construct of claim 4, wherein the tissue specific promoter is a bone specific promoter.

5. The polynucleotide construct of claim 1, further comprising at least one selectable marker.

6. The polynucleotide construct of claim 1 , comprising a polynucleotide sequence encoding a constitutively active ligand independent FGFR2c wherein in said sequence cysteine at position 342 is substituted with tyrosine.

7. A vector comprising a construct comprising a polynucleotide sequence encoding an active variant of FGFR2c and a polynucleotide sequence encoding at least one FGFR2c specific ligand.

8. The vector of claim 7, further comprising a promoter operatively linked to the polynucleotide encoding the active variant of FGFR2c.

9. The vector of claim 7, wherein the construct comprises a polynucleotide encoding a constitutively active FGFR2c stable dimer.

10. The vector of claim 8, wherein the promoter is tissue specific.

11. The vector of claim 10, wherein the tissue specific promoter is a bone-specific promoter.

12. The vector of claim 7, further comprising at least one selectable marker.

13. The vector of claim 7, comprising a polynucleotide sequence encoding a constitutively active ligand independent FGFR2c wherein in said sequence cysteine at position 342 is substituted with tyrosine.

14. The vector of claim 7, wherein the vector is a plasmid or a virus.

15. The vector of claim 14, wherein the virus is selected from the group consisting of: adenoviruses, retroviruses or lentiviruses.

16. A host cell comprising a vector comprising a polynucleotide sequence encoding an active variant of FGFR2c and a polynucleotide sequence encoding at least one FGFR2c specific ligand.

17. The host cell of claim 16, wherein the vector further comprises a promoter operatively linked to the polynucleotide encoding the active variant of FGFR2c.

18. The host cell of claim 17, wherein the promoter is tissue specific.

19. The host cell of claim 18, wherein the tissue specific promoter is a bone-specific promoter.

20. The host cell of claim 16, wherein the vector further comprises at least one selectable marker.

21. The host cell of claim 16, comprising a vector comprising a polynucleotide sequence encoding a constitutively active ligand independent FGFR2c wherein in said sequence cysteine at position 342 is substituted with tyrosine.

22. The host cell of claim 16, wherein the vector is a plasmid or a virus.

23. The host cell of claim 22, wherein the virus is selected from the group consisting of: adenoviruses, retroviruses or lentiviruses.

24. The host cell of claim 16, wherein said cell is capable of expressing the active FGFR2c.

25. The host cell of claim 16, capable of expressing a constitutively active ligand independent FGFR2c.

26. The host cells of claim 25, wherein the constitutively active ligand-independent FGFR2c is a stable dimer.

27. The host cell of claim 16, wherein the cell is eukaryotic.

28. The host cell of according to claim 27, wherein the cell may be is selected from the group consisting of: human embryonic stem cell and human mesenchymal cell.

29. A pharmaceutical composition for regulating bone or cartilage growth or organization comprising as an active ingredient a vector comprising the polynucleotide sequence encoding an active variant of FGFR2c.

30. The pharmaceutical composition of claim 29, further comprising a vector comprising the polynucleotide sequence encoding an FGFR2c specific ligand.

31. The pharmaceutical composition of claim 29, further comprising a physiologically acceptable carrier, diluent, or stabilizer.

32. The pharmaceutical composition of claim 29, further incorporated into liposomes.

33. A method for treating a subject suffering from bone and cartilage defects, disorders or diseases, comprising the step of treating said subject with a therapeutically effective amount of a vector comprising a polynucleotide sequence encoding an active FGFR2c.

34. The method of claim 33, wherein the vector comprises a polynucleotide sequence encoding a constitutively active ligand-independent FGFR2c.

35. The method of claim 34, wherein the constitutively active ligand-independent FGFR2c is a stable dimer.

36. The method of claim 33, further comprising a vector comprising a polynucleotide sequence encoding an FGFR2c specific ligand.

37. The method of claim 33, wherein the vector is a plasmid or a virus.

38. The method of claim 33, wherein the bone or cartilage disorders comprise fractures and inborn defects and disorders.

39. The method of claim 33, wherein the bone and cartilage disorder is osteoporosis.

40. The method of claim 33, wherein the bone and cartilage disorder is osteopetrosis.

41. The method of claim 33, wherein the bone and cartilage disorder is osteoarthritis.

42. The method of claim 33, wherein the bone and cartilage disorder is achondroplasia.

43. The method of claim 33, wherein the route of administration is selected from intravenous, intra-articular, intramuscular, subcutaneous, or intra-lesional.

44. A method for treating a subject suffering from bone or cartilage defects, disorders or diseases, comprising the step of treating said subject with a therapeutically effective amount of recombinant cells expressing at least one of the following receptors: an active variant of FGFR2c, a constitutively active ligand-independent FGFR2c, a constitutively active ligand-independent FGFR2c stable dimer.

45. The method of claim 44, wherein the recombinant cells further express an FGFR2c specific ligand.

46. The method of claim 45, wherein the recombinant cells may be selected from the group consisting of: human embryonic stem cells (hES), human mesenchymal cells, human chondroblasts, human fibroblasts.

47. The method of claim 46, wherein the recombinant cells are autologous.

48. The method of claim 44, wherein the route of administration is selected from intravenous, intra-articular, intramuscular, subcutaneous, or intra-lesional.

49. A non-human transgenic animal having an altered gene, the altered gene comprises a DNA encoding a gain of function mutation in FGFR2c, wherein the gain of function mutation results in the expression of an active variant of FGFR2c in the transgenic animal.

50. The non-human transgenic animal of claim 49, wherein the active variant of FGFR2c is a constitutively active ligand independent FGFR2c.

51. The non-human transgenic mammal of claim 50, wherein the constitutively active ligand independent FGFR2c is a stable dimer.
